SEQUENCE LISTING (28 sequences)

SEQ ID NO: 1 - protein, 21 aa, synthetic construct
MEWSWVFLFFLSVTTGVHSDI

SEQ ID NO: 2 - protein, 10 aa, synthetic construct
EQKLISEEDL

SEQ ID NO: 3 - protein, 46 aa, synthetic construct
AKPTTTPAPRPPTPAPTIASQPLSLRPEACRPAAGGAVHTRGLDFA

SEQ ID NO: 4 - protein, 110 aa, synthetic construct
PRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 6 - protein, 120 aa, synthetic construct
SQVQLVQSGAEVKKPGSSVKVSCKASGGTFSSYAISWVRQAPGQGLEWMGGIIPIFGTANYAQKFQGRVTITADKSTSTAYMDLSSLTSEDTAVYYCARDRGNSADFDSWGQGTLVTVSS

SEQ ID NO: 7 - protein, 108 aa, synthetic construct
DIVMTQSPSSLSASVGDRVTITCRASRDINRWLAWYRRKPGKAPELLIYAASDLKHGVPSRFSGSGSGTDFTLTISSLEPEDFATYYCQQGDSWPFTFGRGTKLEIKR

SEQ ID NO: 8 - protein, 121 aa, synthetic construct
SQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSS

SEQ ID NO: 9 - protein, 110 aa, synthetic construct
QSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLG

SEQ ID NO: 10 - protein, 121 aa, synthetic construct
SQVQLLESHWVGTTSYAQNGKPGASVRSEDTAVYYQRDTSTSTVYMELCACKEHATSYYIFSPGQIVAGTFGLEWASGYSFVKMGIIRVSSSLRAEDGRVRQNPSVTMTYWGQGTLVTVSS

SEQ ID NO: 11 - protein, 128 aa, synthetic construct
QSALDRDSSSTLVFGGFSGSSSEVVSGTLRPGQSITWYTITQPASVSLSKRGPSGVPTQPASVSLYNYVSGSKSGNYYCCTGTSSMIYPKLSYQATYSKRGPSGVPSGLQGEDEAQHPGKAGQLTVLG

SEQ ID NO: 12 - protein, 131 aa, synthetic construct
SQVQLVESGGGVVSRDNSKNTVSCAASGFISYDGEDTAVYYYLQMNSLRQPGRSLRSNKYYADSVKGRAFSSYGMHWVRQAPGFISYDGEDGLEWVALGKFTIVLCARDLFGGGDVLRDSWGQGTLVTVSS

SEQ ID NO: 13 - protein, 108 aa, synthetic construct
DIQMTQSPSPSRFSGSASQPEDFATYYCQQSYSTLFTAPKLLIYAASQSISSYFTLTISSSLSASVGDLNWYPGKGTDQGSFGPTITCRKSGVLRVSLQQGTKVEIKR

SEQ ID NO: 14 - protein, 119 aa, synthetic construct
SQVQLVESGGGFVLNWVRLAPGKGFISRDNSKNTLYLQMNSLYYDSVKGRFYLVQPGGANTRVEDTAVYSGISGSGGLSLTFEWVTRLSCAASDSTCANLWTAAGIDYWGQGTLVTVSS

SEQ ID NO: 15 - protein, 111 aa, synthetic construct
QSALTQPASVASHRFSTTNNDESGSKISCTASTLGTSSDIGKSGSMYDVSSRPSGAPKFIYDRVSWYQQHPVFGLQADLTISGNTGSPGQSITADYYCNSYGGGTKLTVLG

SEQ ID NO: 16 - protein, 115 aa, synthetic construct
SQVQLVESGNSTAVYDVWLVCAACAKEVWGGLVRKNTLYISGSGGSTYYADQMNSLRAEYVQPEWSVGSSGFSGFLWSRLSTSYAMSLEKGRFTISRDDQAPGKGGKGTIVTVSS

SEQ ID NO: 17 - protein, 113 aa, synthetic construct
NIVMTQSPSTYLTISNLQPVGDRVITCRAKPGQSLSASVGDRVTWYQVPSRFSGSGSHSAPKLLIYFNTSLQEDSFPWSGGTTQSIRDFTLATYYCQQYHGALGHGTKLEIKR

SEQ ID NO: 18 - protein, 512 aa, synthetic construct
SQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLGGGGGSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLGGGGGSGGGGSGGGGSSQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSS

SEQ ID NO: 19 - protein, 387 aa, synthetic construct
SQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSGGGGSSQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLG

SEQ ID NO: 20 - protein, 778 aa, unidentified
SQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLGGGGGSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLGGGGGSGGGGSGGGGSSQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSGGGGSSQVTLRESGPGLVRPSQTLSLTCTVSGGSIDSGGHYWSWIRQHPGKGLEWIGSIYHSGNTYYNPSLKSRVTMSVDTSKNQFSLKLTSVTAADTAIYYCARDIPHYFEPAYWGQGTLVTVSSGGGGSGGGGSGGGGSQSALTQPASASGSPGQSVTISCTGTSSDVGGYNYVSWYQQHPGKAPKLMIYEVSNRPSGVSNRFSGSKSGNTASLTISGLQAEDEADYYCAIWHSSAWVFGGGTKLTVLG

SEQ ID NO: 21 - DNA, 37 bp, synthetic construct
  source 1..37: mol_type = other DNA
cagctagcctcgagaccatggagtggtcctgggtgtt

SEQ ID NO: 22 - DNA, 44 bp, synthetic construct
  source 1..44: mol_type = other DNA
gctggggtagggggtctgggagcgggggtggttgtgggcttggc

SEQ ID NO: 23 - DNA, 44 bp, synthetic construct
  source 1..44: mol_type = other DNA
gccaagcccacaaccacccccgctcccagaccccctaccccagc

SEQ ID NO: 24 - DNA, 46 bp, synthetic construct
  source 1..46: mol_type = other DNA
ctggatccgcggccgcgaattcctattatctgggaggcagggcctg

SEQ ID NO: 25 - DNA, 24 bp, synthetic construct
  source 1..24: mol_type = other DNA
gacaccagactaagaacctagaac

SEQ ID NO: 26 - DNA, 24 bp, synthetic construct
  source 1..24: mol_type = other DNA
ctcaaagtagacggcatcgcagct

SEQ ID NO: 27 - DNA, 24 bp, synthetic construct
  source 1..24: mol_type = other DNA
tagacggcatcgcagcttggatac

SEQ ID NO: 28 - DNA, 24 bp, synthetic construct
  source 1..24: mol_type = other DNA
caccagctgaagcctatagagtac

(19) United States
(12) Patent Application Publication
(10) Pub. No.: US 2026/0295044 A1
(43) Pub. Date: Oct. 1, 2026

(54) CHIMERIC ANTIGEN RECEPTOR TARGETING BCMA AND USE THEREOF

(71) Applicant: SHENZHEN CELL VALLEY BIOMEDICAL CO., LTD., Shenzhen, Guangdong (CN)

(72) Inventors: Jianxun WANG, Shenzhen, Guangdong (CN); Xiaorui LI, Shenzhen, Guangdong (CN); Yaru FENG, Shenzhen, Guangdong (CN); Fengqin SHANG, Shenzhen, Guangdong (CN); Zhuoying YU, Shenzhen, Guangdong (CN); Zhiru SONG, Shenzhen, Guangdong (CN); Jiansong TONG, Shenzhen, Guangdong (CN); Ying LIU, Shenzhen, Guangdong (CN)

(73) Assignee: SHENZHEN CELL VALLEY BIOMEDICAL CO., LTD., Shenzhen, Guangdong (CN)

(21) Appl. No.: 19/163,185

(22) PCT Filed: Jul. 11, 2023
(86) PCT No.: PCT/CN2023/106789

§ 371 Date: Sep. 8, 2025

(30) Foreign Application Priority Data
Mar. 8, 2023 (CN) 202310214870.8

(51) Int. Cl. A61K 40/41; A61K 40/11; A61K 40/31; A61P 35/00; C07K 14/705; C07K 16/28

(52) U.S. Cl. CPC A61K 40/41; A61K 40/11; A61K 40/31; A61P 35/00; C07K 14/7051; C07K 14/70517; C07K 14/70521; C07K 14/70578; C07K 16/2878; C07K 2317/24; C07K 2317/622

(57) ABSTRACT

Provided is a BCMA-targeting chimeric antigen receptor. The receptor comprises a humanized BCMA-targeting ScFv structure, wherein the heavy chain variable region ScFv-VH and the light chain variable region ScFv-VL of the ScFv structure are connected by one or more G4S sequences. A new second-generation CAR-T cell targeting BCMA is successfully constructed by means of transduction by using a retroviral vector. In vitro and in vivo experiments show that after stimulation with BCMA positive tumor cells, the BCMA CAR 31-T cells can be effectively activated to secrete cytokines such as IFN-γ and TNF-α, thereby promoting the apoptosis of tumor cells. The BCMA CAR 31-T cells can effectively and specifically kill BCMA positive tumor cells.

### TECHNICAL FIELD

The present invention relates to the field of cellular immunotherapy for tumors, and in particular to BCMA-targeting chimeric antigen receptor.

### BACKGROUND ART

Multiple myeloma (MM) is a malignant tumor with abnormal proliferation of terminally differentiated plasma cells and is the second most common hematological malignancy. In recent years, with the development of new therapeutic drugs such as proteasome inhibitors and immunomodulators, the prognosis of patients has been significantly improved. However, MM is still an incurable disease, and almost all patients will eventually face the risk of recurrence and drug resistance. Therefore, new treatment regimens are crucial for these patients. Immunotherapy represented by chimeric antigen receptor T (CAR-T) cells has shown good efficacy in hematological tumors and is expected to become a new and effective treatment for MM.

Unlike traditional T cell activation pathways, the activation of CAR-T cells does not depend on the presentation of MHC. CAR-T cells can recognize and bind to tumor target cell surface antigens through extracellular single-chain variable fragments (ScFv). In the initial design, the extracellular antigen binding domain and the intracellular signal transduction domain are connected through a transmembrane domain (first-generation CAR), which can directly induce T cell activation after recognizing and binding to the antigen. The antigen binding domain binds to the ligand on the cell surface to provide the first signal. Once bound to the ligand, the intracellular co-stimulatory molecules are immediately activated to provide the second signal. The first signal and the second signal are transmitted to the intracellular activation domain to activate CAR-T and exert anti-tumor activity. Studies have found that the first-generation CAR structure has almost no tumor clearance effect and no proliferation activity. Therefore, researchers have constructed a second-generation CAR containing co-stimulatory domains such as CD28 or 4-1BB. The second-generation CAR has stronger anti-tumor activity. CARs that integrate more than one intracellular co-stimulatory domain (such as CD28, OX40, 4-1BB, CD27, etc.) become third-generation CARs. The fourth generation CAR is a CAR that can secrete cytokines such as IL-12 and express cell surface markers such as co-stimulatory ligands. Currently, the second-generation CAR structure shows better safety and clinical efficacy, so it is more widely used in clinical practice.

A typical CAR-T cell manufacturing requires drawing peripheral blood from the patient, isolating peripheral blood mononuclear cells (PBMC), stimulating and activating T cells, and then using genetic engineering methods to express CAR on the cell surface that can specifically recognize tumor target antigens. The successfully prepared CAR-T cells are amplified in large quantities and then infused back into the patient's body to exert anti-tumor function. The production of CAR-T cells generally takes 10-14 days. Since 2017, five CAR-T products have been approved by the FDA for the treatment of hematological malignancies. Among them, Abecma targeting BCMA was approved for marketing this year and is the first CAR-T cell product for the treatment of MM.

Identification of tumor-specific antigens is key to the success of CAR-T cell therapy. First, the antigen must be expressed on the surface of tumor cells. Second, the antigen must be uniformly expressed on tumor cells and ideally should be required for tumor survival. Most importantly, the target antigen must not be expressed in related healthy tissues to avoid potential on-target off-tumor toxicity.

BCMA, also known as CD269 or TNFRSF17, is a type III transmembrane glycoprotein of 27 kDa expressed by mature B lymphocytes, plasma cells, and most patients with multiple myeloma. BCMA can bind to a variety of ligands, including BAFF (B cell activating factor) and APRIL (A proliferation-inducing ligand), mediating cell survival through downstream NF-κB and MAPK/JNK signaling pathways. BCMA is a member of the tumor necrosis factor family and plays a crucial role in supporting plasma cell differentiation and survival. It is selectively expressed on normal plasma cells and MM cells, and BCMA expression is not detected in cells from other tissues. BCMA, along with transmembrane activator, calcium regulator, and cyclophilin ligand interactor (TACI), is also a receptor for proliferation-inducing ligand (APRIL). In multiple myeloma, the APRIL/BCMA pathway plays a key role in supporting growth, drug resistance, and an immune-compromised environment. Due to its unique expression on plasma cells and its important role in multiple myeloma, BCMA has become an ideal target for the treatment of multiple myeloma.

Although BCMA CAR-T cells targeting MM have been reported, clinical trials of novel CAR-T cells are still needed to advance the treatment of MM.

### SUMMARY OF THE INVENTION

In order to solve the above problems, the present invention provides a BCMA-targeting chimeric antigen receptor, wherein the receptor comprises a humanized BCMA-targeting ScFv structure, the heavy chain variable region ScFv-VH and the light chain variable region ScFv-VL of the ScFv structure are connected by one or more G4S sequences; the amino acid sequence of the heavy chain variable region ScFv-VH has at least 90% homology with the following SEQ ID NO.8, preferably at least 95% homology, and more preferably at least 98% homology, SEQ ID NO.8:

SQVTLRESGPGLVRPSQTLSLTCTVSGGSIDSGGHYWSWIRQHPG
KGLEWIGSIYHSGNTYYNPSLKSRVTMSVDTSKNQFSLKLTSVTA
ADTAIYYCARDIPHYFEPAYWGQGTLVTVSS;

the amino acid sequence of the light chain variable region ScFv-VL has at least 90% homology with the following SEQ ID NO.9, preferably at least 95% homology, and more preferably at least 98% homology: SEQ ID NO.9:

QSALTQPASASGSPGQSVTISCTGTSSDVGGYNYVSWYQQHPGKA
PKLMIYEVSNRPSGVSNRFSGSKSGNTASLTISGLQAEDEADYYC
AIWHSSAWVFGGGTKLTVLG.

In one embodiment, the humanized BCMA-targeting ScFv structure of the receptor is ScFv-VH-(G4S) n-ScFv-VL, wherein n is an integer greater than or equal to 1, preferably 3 or 4.

In one embodiment, the humanized BCMA-targeting ScFv structure of the receptor is ScFv-VH-(G4S) 3-ScFv-VL, the amino acid sequence of the heavy chain variable region ScFv-VH is SEQ ID NO.8; the amino acid sequence of the light chain variable region ScFv-VL is SEQ ID NO.9.

In one embodiment, the humanized BCMA-targeting ScFv structure of the receptor is ScFv-VH-(G4S) n-ScFv-VL-(G4S) n-ScFv-VL-(G4S) n-ScFv-VH, wherein n is an integer greater than or equal to 1, preferably 3 or 4.

In one embodiment, the humanized BCMA-targeting ScFv structure of the receptor is ScFv-VH-(G4S) 3-ScFv-VL-(G4S) 4-ScFv-VL-(G4S) 3-ScFv-VH, the amino acid sequence of the heavy chain variable region ScFv-VH is SEQ ID NO.8; the amino acid sequence of the light chain variable region ScFv-VL is SEQ ID NO.9.

In one embodiment, t the receptor comprises an upstream signal peptide and a myc tag for detection; a humanized BCMA-targeting ScFv structure comprising a heavy chain variable region and a light chain variable region; a CD8 hinge-transmembrane domain; a CD28 or 4-1BB synergistic activation domain and a CD3ζ intracellular signaling domain, which are sequential tandem.

In one embodiment, the present invention provides a BCMA-targeting chimeric antigen receptor T cell, wherein it expresses the above-mentioned chimeric antigen receptor.

In one embodiment, the present invention provides a drug for treating tumors, wherein it comprises the above-mentioned chimeric antigen receptor T cell.

In one embodiment, the present invention provides the use of the above-mentioned chimeric antigen receptor for preparing chimeric antigen receptor T cells and treating tumor.

In one embodiment, the tumor is a surface BCMA-positive tumor.

In one embodiment, the tumor is multiple myeloma.

In one embodiment, the present invention provides the use of the above-mentioned chimeric antigen receptor, wherein a gene fragment encoding the chimeric antigen receptor is inserted into a viral expression vector, packaged into viral vector particles, infecting human T cells to prepare chimeric antigen receptor T cells for the treatment of surface BCMA-positive tumors.

In the present invention, we successfully constructed pMFG-BCMA CAR15, pMFG-BCMA CAR16, pMFG-BCMA CAR17, pMFG-BCMA CAR18, pMFG-BCMA CAR19 and pMFG-BCMA CAR20 plasmids and transiently transfected Phoenix-ECO cells, with transfection efficiencies all higher than 50%. The PG13 cell line was transduced using transiently transfected retroviral vector particles, with transduction efficiencies all higher than 95%. pPCR detection showed that the retroviral vector particles had a high viral titer, with the highest titer all higher than 1×10⁷ copies/mL, indicating that the PG13 cell line stably expressing and producing the BCMA CAR retroviral vector was successfully prepared. The successfully prepared amphotropic retroviral vectors BCMA CAR15, BCMA CAR16, BCMA CAR17, BCMA CAR18, BCMA CAR19 and BCMA CAR20 were used to transduce human primary T cells and detect the expression of CAR, indicating that we have successfully prepared BCMA CAR-T cells.

In the in vitro tumor killing experiment, the present invention uses flow cytometry to detect apoptosis of target cells and luciferase bioluminescence to detect survival of target cells, among which BCMA CAR16-T shows relatively better anti-tumor ability. However, since the killing effect is not as good as that of the positive control group, it shows that the BCMA CAR16 we screened can be further optimized to improve the binding ability to the target antigen.

Since BCMA has a trimer structure and a small protein molecular weight of 32.3 kDa, we concatenated the ScFv structure of BCMA CAR16 to design BCMA CAR31, BCMA CAR32 and BCMA CAR33 to increase the flexibility of the ScFv region of the extracellular antigen binding domain, better capture antigens, and improve the ability to kill tumors.

BCMA CAR31, BCMA CAR32 and BCMA CAR33 were prepared into amphotropic retroviral vectors and transduced into primary human T cells. The transduction efficiency was over 50%, and qPCR detection showed successful integration into the T cell genome.

In the in vitro tumor killing experiment, incucyte real-time dynamic live cell imaging screened out the BCMA CAR31-T cells with the best killing effect, and further compared them with BCMA CAR16-T cells. CD69 is a marker of T cell activation. We found that BCMA CAR31-T and BCMA CAR16-T cells had no obvious self-activation phenomenon when there was no tumor cell stimulation. BCMA CAR-T cells could be effectively activated under tumor cell stimulation and could play an anti-tumor role.

In order to determine the anti-tumor ability of BCMA CAR31-T cells in vitro, we selected RPMI-gfp-luc tumor cells expressing human BCMA. The results of luciferase bioluminescence assay to detect the survival of target cells showed that compared with the BCMA CAR16-T group, BCMA CAR31-T cells had stronger killing ability against two different BCMA-positive tumor cells at different effector-target ratios, indicating that the killing ability of BCMA CAR31-T cells after structural optimization on tumor cells was improved.

In order to verify the antigen specificity of BCMA CAR31-T cells in anti-tumor effects, BCMA CAR31-T cells were co-cultured with different types of tumor cell lines K562-hBCMA-gfp, PMI-gfp-luc, K562-cBCMA and K562. The results showed that compared with the BCMA CAR16-T group, BCMA CAR31-T cells had stronger killing power against two different BCMA-positive tumor cells at different effector-target ratios. However, BCMA CAR31-T showed no difference in killing power with Pan-T for non-human BCMA-expressing K562-cBCMA and BCMA-negative K562 cells. This result shows that BCMA CAR31-T cells have BCMA antigen specificity in killing tumor cells.

When CAR-T cells bind to tumor antigens, they recruit other immune cells, release a large amount of cytokines, and produce anti-tumor immune responses. In the cytokine secretion experiment, compared with BCMA CAR16-T, BCMA CAR31-T cells secrete more pro-inflammatory cytokines TNF-α, IFN-γ, IL-6, IL-17A, and aFasL under the stimulation of tumor cells, thereby promoting the apoptosis of tumor cells.

To generate a strong and lasting anti-tumor immune response, it is necessary not only to induce cytotoxicity and cytokine production, but also to stimulate the proliferation of CAR-T cells. We found through CFSE assay that BCMA CAR31-T cells proliferated faster than BCMA CAR16-T cells.

In addition, the present invention establishes an NPG mouse xenograft tumor model to confirm the anti-tumor ability of BCMA CAR31-T cells in vivo. Two days after the second tail vein injection of BCMA CAR31-T, the secretion of IFN-γ cytokines was significantly enhanced compared with BCMA CAR16-T, indicating that BCMA CAR31-T cells were better activated; the mice were in general good condition and did not show symptoms of cytokine release syndrome such as fever, nausea, and vomiting. During the continuous observation of mouse tumor changes, the tumor signal in the BCMA CAR31-T group was significantly weakened than that of BCMA CAR16-T, showing a more effective anti-tumor ability; flow cytometry was used to continuously detect the content of T cells in the peripheral blood of mice. The results showed that until the 37th day after tumor inoculation, the content of BCMA CAR31-T cells was higher than that of BCMA CAR16-T cells, indicating that BCMA CAR31-T cells continued to survive longer in mice and had a more lasting anti-tumor ability.

Therefore, in the present invention, a new second-generation CAR-T cell targeting BCMA was successfully constructed by retroviral vector transduction. In vitro experiments showed that BCMA-positive tumor cells can effectively activate BCMA CAR31-T cells after stimulation, secrete cytokines such as IFN-γ and TNF-α, and promote tumor cell apoptosis. BCMA CAR31-T cells can effectively and specifically kill BCMA-positive tumor cells. BCMA CAR31-T cells have good proliferation ability in vitro. Further in vivo anti-tumor experiments showed that BCMA CAR31-T cells can be quickly activated to secrete IFN-γ and have a certain tumor effect. Therefore, the new second-generation CAR-T cells targeting BCMA have efficient and specific anti-tumor activity and may become a new therapy for the clinical treatment of MM.

### BRIEF DESCRIPTION OF THE DRAWINGS

In order to more clearly illustrate the technical solutions in the embodiments of the present application, the drawings required for use in the embodiments will be briefly introduced below. Obviously, the drawings described below are only some embodiments recorded in the present application. For ordinary technicians in this field, other drawings can be obtained based on these drawings without creative work.

FIG. 1 is an experimental scheme diagram of the present invention;

FIG. 2 is a schematic diagram of the structure of the BCMA CAR of the present invention;

FIG. 3 is a diagram of the preparation scheme of BCMA CAR retroviral vector plasmid;

FIG. 4 is a technical route diagram of a BCMA CAR retrovirus vector packaging;

FIG. 5 is a technical route diagram of the preparation of BCMA CAR-T cells;

FIG. 6 is a technical route diagram of BCMA CAR-T's in vitro anti-tumor function;

FIG. 7 is a graph showing the efficiency results of a flow cytometry detection of BCMA CAR-T cells to kill K562-hBCMA-gfp cells;

FIG. 8 is a graph showing BCMA CAR-T cells killing RPMI-gfp-luc cells efficiency detection (n=3) (compared with bb2121 CAR-T cells, *P<0.05) result;

FIG. 9 is a schematic diagram of the structure of an optimized BCMA CAR;

FIG. 10 is a Gel electrophoresis diagram of enzyme digestion identification, wherein A: pMFG-BCMA CAR31 and pMFG-BCMA CAR32 enzyme digestion identification fragment; B: pMFG-BCMA CAR33 enzyme digestion identification fragment;

FIG. 11 is a graph showing the transduction efficiency of optimized BCMA CAR dual-tropic retroviral vector particles transducing primary human T cells (n=3) (ns: no statistical difference);

FIG. 12 is a diagram of an optimized BCMA CAR-T cell in-vitro killing experiment scheme;

FIG. 13 is a graph showing the real-time fluorescence monitoring results of the optimized BCMA CAR-T cells killing K562-hBCMA-gfp in vitro (n=3);

FIG. 14 is a graph showing the results of CAR-T cell CD69 expression (n=3), wherein A: flow cytometry of CD69 expression on the surface of BCMA CAR-T cells; B: histogram of CD69 expression on the surface of BCMA CAR-T cells without tumor cell stimulation; C: histogram of CD69 expression on the surface of BCMA CAR-T cells under BCMA+ tumor cell stimulation (compared with BCMA CAR16-T cells, *P<0.05, ns: no statistical difference);

FIG. 15 is a graph showing the results of detecting the in vitro killing ability of BCMA CAR-T cells by luciferase bioluminescence assay (n=3) (compared with BCMA CAR16-T cells, *P<0.05, **P<0.01, ***P<0.001);

FIG. 16 is a graph showing the results of the efficiency test of BCMA CAR-T cells killing different tumor cells (n=3), (compared with BCMA CAR16-T cells, *P<0.05, **P<0.01, ***P<0.001, ****P<0.0001, ns: no statistical difference);

FIG. 17 is a graph showing the secretion level of BCMA CAR-T cell factors (n=3) (compared with BCMA CAR16-T cells, *P<0.05, **P<0.01, ***P<0.001, ****P<0.0001);

FIG. 18 is a graph showing the results of BCMA CAR-T cell proliferation ability detection;

FIG. 19 is a diagram of the BCMA CAR-T in vivo anti-tumor experimental scheme;

FIG. 20 is a graph showing the results of living body imaging of a mouse xenograft tumor model (n=6), A: mouse living body imaging picture; B: statistical graph of average signal intensity of mouse tumors (ns: no statistical difference);

FIG. 21 is a graph showing the results of living body imaging of a mouse xenograft tumor model (n=6), A: mouse living body imaging picture; B: statistical graph of average signal intensity of mouse tumors (compared with BCMA CAR16-T cells, *P<0.05, **P<0.01, ***P<0.001).

### DETAILED DESCRIPTION OF THE EMBODIMENTS

In order to enable those skilled in the art to better understand the technical solutions in this application, the present invention will be further described below in conjunction with embodiments. Obviously, the described embodiments are only part of the embodiments of this application, rather than all of the embodiments. Based on the embodiments in this application, all other embodiments obtained by ordinary technicians in this field without creative work should fall within the scope of protection of this application.

It should be understood that the disclosed invention is not limited only to the specific method, scheme and material of description, because these all can change. It should also be understood that the terminology used herein is only for the purpose of describing specific embodiment scheme, rather than being intended to limit the scope of the present invention, and the scope of the present invention is only limited to the appended claims.

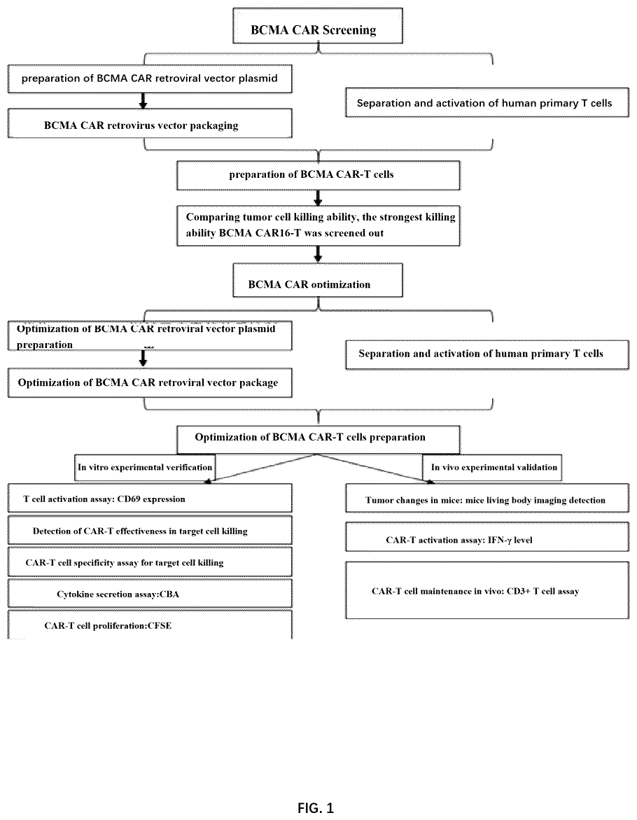

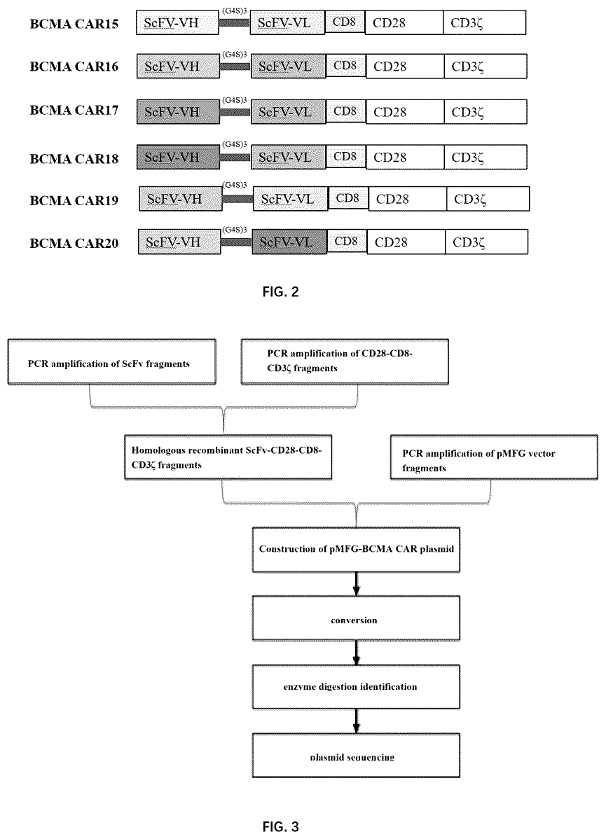

Through the phage display method, six new human ScFv structures targeting BCMA were screened out. The specific experimental scheme is shown in FIG. 1. In order to verify whether these six ScFvs targeting BCMA can effectively and specifically recognize BCMA-positive tumor cells, we used CD8 as a transmembrane domain, CD28 and CD3ζ as intracellular stimulation domains, and designed and constructed a second-generation human BCMA CAR structure, see FIG. 2; BCMA CAR-T cells were prepared by retroviral vectors. The in vitro killing of tumor cells experiment screened out the BCMA CAR16-T cells with the strongest ability to kill tumor cells. We further connected the ScFv of BCMA CAR16 in series to further improve the antigen binding ability to BCMA. In the in vitro experiment, tumor cells expressing human BCMA antigen were used as target cells to detect the activation of BCMA CAR-T after optimization, verify the killing specificity and effectiveness of BCMA CAR-T cells on target cells, detect the proliferation ability and cytokine secretion level of BCMA CAR-T, and further verify it in the in vivo xenograft tumor model to detect the secretion of cytokines and anti-tumor ability, as well as the maintenance of CAR-T cells in vivo after treatment.

In the present invention, the amino acid sequence is as follows:

SP amino acid sequence:
(SEQ ID NO. 1)
MEWSWVFLFFLSVTTGVHSDI;
Myc amino acid sequence:
(SEQ ID NO. 2)
EQKLISEEDL;
CD8 amino acid sequence:
(SEQ ID NO. 3)
AKPTTTPAPRPPTPAPTIASQPLSLRPEACRPAAGGAVHTRGLDFA;
CD28 amino acid sequence :
(SEQ ID NO. 4)
PRKIEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPRDFA
AYRS;
CD3Z amino acid sequence:
(SEQ ID NO. 5)
RVKFSRSADAPAYQQGQNQLYNELNLGRREEYDVLDKRRGRDPEMGGKPQR
RKNPQEGLYNELQKDKMAEAYSEIGMKGERRRGKGHDGLYQGLSTATKDTY
DALHMQALPPR;
BCMA CAR15 ScFv-VH amino acid sequence:
(SEQ ID NO. 6)
SQVQLVQSGAEVKKPGSSVKVSCKASGGTFSSYAISWVRQAPGQGLEWMGGII
PIFGTANYAQKFQGRVTITADKSTSTAYMDLSSLTSEDTAVYYCARDRGNSAD
FDSWGQGTLVTVSS;
BCMA CAR15 ScFv-VL amino acid sequence:
(SEQ ID NO. 7)
DIVMTQSPSSLSASVGDRVTITCRASRDINRWLAWYRRKPGKAPELLIYAASD
LKHGVPSRFSGSGSGTDFTLTISSLEPEDFATYYCQQGDSWPFTFGRGTKLEI
KR;
BCMA CAR16 ScFv-VH amino acid sequence:
(SEQ ID NO. 8)
SQVTLRESGPGLVRPSQTLSLTCTVSGGSIDSGGHYWSWIRQHPGKGLEWIGSI
YHSGNTYYNPSLKSRVTMSVDTSKNQFSLKLTSVTAADTAIYYCARDIPHYFE
PAYWGQGTLVTVSS;
BCMA CAR16 ScFv-VL amino acid sequence:
(SEQ ID NO. 9)
QSALTQPASASGSPGQSVTISCTGTSSDVGGYNYVSWYQQHPGKAPKLMIYE
VSNRPSGVSNRFSGSKSGNTASLTISGLQAEDEADYYCAIWHSSAWVFGGGTK
LTVLG;
BCMA CAR17 ScFv-VH amino acid sequence:
(SEQ ID NO. 10)
SQVQLLESHWVGTTSYAQNGKPGASVRSEDTAVYYQRDTSTSTVYMELCACK
EHATSYYIFSPGQIVAGTFGLEWASGYSFVKMGIIRVSSSLRAEDGRVRQNPS
VTMTYWGQGTLVTVSS;
BCMA CAR17 ScFv-VL amino acid sequence:
(SEQ ID NO. 11)
QSALDRDSSSTLVFGGFSGSSSEVVSGTLRPGQSITWYTITQPASVSLSKRGPSG
VPTQPASVSLYNYVSGSKSGNYYCCTGTSSMIYPKLSYQATYSKRGPSGVPSG
LQGEDEAQHPGKAGQLTVLG;
BCMA CAR18 ScFv-VH amino acid sequence:
(SEQ ID NO. 12)
SQVQLVESGGGVVSRDNSKNTVSCAASGFISYDGEDTAVYYYLQMNSLRQPG
RSLRSNKYYADSVKGRAFSSYGMHWVRQAPGFISYDGEDGLEWVALGKFTIV
LCARDLFGGGDVLRDSWGQGTLVTVSS;
BCMA CAR18 ScFv-VL amino acid sequence:
(SEQ ID NO. 13)
DIQMTQSPSPSRFSGSASQPEDFATYYCQQSYSTLFTAPKLLIYAASQSISSYFTL
TISSSLSASVGDLNWYPGKGTDQGSFGPTITCRKSGVLRVSLQQGTKVEIKR;
BCMA CAR19 ScFv-VH amino acid sequence:
(SEQ ID NO. 14)
SQVQLVESGGGFVLNWVRLAPGKGFISRDNSKNTLYLQMNSLYYDSVKGRFY
LVQPGGANTRVEDTAVYSGISGSGGLSLTFEWVTRLSCAASDSTCANLWTAAG
IDYWGQGTLVTVSS;
BCMA CAR19 ScFv-VL amino acid sequence:
(SEQ ID NO. 15)
QSALTQPASVASHRFSTTNNDESGSKISCTASTLGTSSDIGKSGSMYDVSSRPSG
APKFIYDRVSWYQQHPVFGLQADLTISGNTGSPGQSITADYYCNSYGGGTKLT
VLG;
BCMA CAR20 ScFv-VH amino acid sequence:
(SEQ ID NO. 16)
SQVQLVESGNSTAVYDVWLVCAACAKEVWGGLVRKNTLYISGSGGSTYYAD
QMNSLRAEYVQPEWSVGSSGFSGFLWSRLSTSYAMSLEKGRFTISRDDQAPG
KGGKGTIVTVSS;
BCMA CAR20 ScFv-VL amino acid sequence:
(SEQ ID NO. 17)
NIVMTQSPSTYLTISNLQPVGDRVITCRAKPGQSLSASVGDRVTWYQVPSRES
GSGSHSAPKLLIYFNTSLQEDSFPWSGGTTQSIRDFTLATYYCQQYHGALGHG
TKLEIKR;
BCMA CAR31 ScFv amino acid sequence:
(SEQ ID NO. 18)
SQVTLRESGPGLVRPSQTLSLTCTVSGGSIDSGGHYWSWIRQHPGKGLEWIGS
IYHSGNTYYNPSLKSRVTMSVDTSKNQFSLKLTSVTAADTAIYYCARDIPHYF
EPAYWGQGTLVTVSSGGGGSGGGGSGGGGSQSALTQPASASGSPGQSVTISC
TGTSSDVGGYNYVSWYQQHPGKAPKLMIYEVSNRPSGVSNRFSGSKSGNTAS
LTISGLQAEDEADYYCAIWHSSAWVFGGGTKLTVLGGGGGSGGGGSGGGGS
GGGGSQSALTQPASASGSPGQSVTISCTGTSSDVGGYNYVSWYQQHPGKAPK
LMIYEVSNRPSGVSNRFSGSKSGNTASLTISGLQAEDEADYYCAIWHSSAWVF
GGGTKLTVLGGGGGSGGGGSGGGGSSQVTLRESGPGLVRPSQTLSLTCTVSG
GSIDSGGHYWSWIRQHPGKGLEWIGSIYHSGNTYYNPSLKSRVTMSVDTSKN
QFSLKLTSVTAADTAIYYCARDIPHYFEPAYWGQGTLVTVSS;
BCMA CAR32 ScFv amino acid sequence:
(SEQ ID NO. 19)
SQVTLRESGPGLVRPSQTLSLTCTVSGGSIDSGGHYWSWIRQHPGKGLEWIGS
IYHSGNTYYNPSLKSRVTMSVDTSKNQFSLKLTSVTAADTAIYYCARDIPHYF
EPAYWGQGTLVTVSSGGGGSGGGGSGGGGSGGGGSSQVTLRESGPGLVRPSQ
TLSLTCTVSGGSIDSGGHYWSWIRQHPGKGLEWIGSIYHSGNTYYNPSLKSR
VTMSVDTSKNQFSLKLTSVTAADTAIYYCARDIPHYFEPAYWGQGTLVTVSS
GGGGSGGGGSGGGGSQSALTQPASASGSPGQSVTISCTGTSSDVGGYNYVSW
YQQHPGKAPKLMIYEVSNRPSGVSNRFSGSKSGNTASLTISGLQAEDEADYY
CAIWHSSAWVFGGGTKLTVLG,
where the underline in the
sequence is (G4S)n;
BCMA CAR33 ScFv amino acid sequence:
(SEQ ID NO. 20)
SQVTLRESGPGLVRPSQTLSLTCTVSGGSIDSGGHYWSWIRQHPGKGLEWIGS
IYHSGNTYYNPSLKSRVTMSVDTSKNQFSLKLTSVTAADTAIYYCARDIPHYF
EPAYWGQGTLVTVSSGGGGSGGGGSGGGGSQSALTQPASASGSPGQSVTISC
TGTSSDVGGYNYVSWYQQHPGKAPKLMIYEVSNRPSGVSNRFSGSKSGNTAS
LTISGLQAEDEADYYCAIWHSSAWVFGGGTKLTVLGGGGGSGGGGSGGGGS
GGGGSQSALTQPASASGSPGQSVTISCTGTSSDVGGYNYVSWYQQHPGKAPK
LMIYEVSNRPSGVSNRFSGSKSGNTASLTISGLQAEDEADYYCAIWHSSAWVF
GGGTKLTVLGGGGGSGGGGSGGGGSSQVTLRESGPGLVRPSQTLSLTCTVSG
GSIDSGGHYWSWIRQHPGKGLEWIGSIYHSGNTYYNPSLKSRVTMSVDTSKN
QFSLKLTSVTAADTAIYYCARDIPHYFEPAYWGQGTLVTVSSGGGGSGGGGS
GGGGSGGGGSSQVTLRESGPGLVRPSQTLSLTCTVSGGSIDSGGHYWSWIRQ
HPGKGLEWIGSIYHSGNTYYNPSLKSRVTMSVDTSKNQFSLKLTSVTAADTAI
YYCARDIPHYFEPAYWGQGTLVTVSSGGGGSGGGGSGGGGSQSALTQPASA
SGSPGQSVTISCTGTSSDVGGYNYVSWYQQHPGKAPKLMIYEVSNRPSGVSN
RFSGSKSGNTASLTISGLQAEDEADYYCAIWHSSAWVFGGGTKLTVLG,
where the underlined sequence is (G4S)n sequence.

### Example 1 Construction of BCMA CAR Retroviral Vector Plasmid

Six ScFv sequences targeting BCMA were screened out and synthesized into pUC57 vector by General Biotech Co., Ltd. Plasmids containing CD28-CD8-CD3ζ sequences and retroviral vector pMFG plasmids can be purchased commercially or prepared by themselves. ScFv fragments and CD28-CD8-CD3ζ fragments are amplified by PCR, and then ScFv-CD28-CD8-CD3 ζ fragments are amplified by homologous recombination. The recombinant target fragment and the vector are double-digested with XhoI and NotI, and then connected to construct a complete pMFG-BCMA CAR plasmid. The specific experimental scheme is shown in FIG. 3.

### I. Experimental Procedure

### 1. Extraction of Vector Plasmid and Target Plasmid

The pUC57-BCMA CAR15, pUC57-BCMA CAR16, pUC57-BCMA CAR17, pUC57-BCMA CAR18, pUC57-BCMA CAR19, pUC57-BCMA CAR20, pMFG-CD8-CD28-CD3ζ and vector plasmid pMFG strains stored at −80° C. were taken out, and 50 μL of each was added to 25 mL of LB liquid medium (final concentration of Amp was 50 μg/mL), and shaken at 37° C., 200 rpm in a shaker for 12-16 hours to a cell density of 3-4× 10⁹/mL. The bacterial precipitate was harvested, centrifuged, suspended, and filtered, and 4 mL of isopropanol was added to the eluent and mixed evenly. Immediately centrifuged at 15000 g for 30 minutes at 4° C., and the supernatant was carefully discarded. Add 2 mL of 75% ethanol to wash the DNA precipitate, centrifuge at 15000 g for 10 min at room temperature, and carefully discard the supernatant. Dry the precipitate for 5-10 min, add an appropriate volume of nuclease-free water, and flick to mix. Measure the DNA concentration and store the plasmid at −20° C. for later use.

### 2. PCR Amplification of Target Fragment

The pUC57-BCMA CAR15, pUC57-BCMA CAR16, pUC57-BCMA CAR17, pUC57-BCMA CAR18, pUC57-BCMA CAR19, and pUC57-BCMA CAR20 plasmids obtained above were amplified by PCR, and each target fragment was about 800 bp. The pMFG-CD8-CD28-CD3ζ plasmid obtained above was amplified by PCR, and the fragment was about 800 bp.

TABLE 1
Primer sequence list table

| Primer | |
|---|---|
| Name | Primer Sequence |
| CAR-For | caGCTAGCctcgagaccATGGAGTGG |
| | TCCTGGGTGTT (SEQ ID NO. 21) |
| CD8-Rev | GCTGGGGTAGGGGGTCTGGGAGCGGG |
| | GGTGGTTGTGGGCTTGGC |
| | (SEQ ID NO. 22) |
| CD8-For | GCCAAGCCCACAACCACCCCCGCTCCC |
| | AGACCCCCTACCCCAGC |
| | (SEQ ID NO. 23) |
| CAR-Rev | ctGGATCCgcggccgcGAATTCctaTT |
| | ATCTGGGAGGCAGGGCCTG |
| | (SEQ ID NO. 24) |

The obtained target fragments were named CAR15-ScFv, CAR16-ScFv, CAR17-ScFv, CAR18-ScFv, CAR19-ScFv, CAR20-ScFv and CD8-CD28-CD3 ζ. The obtained CAR15-ScFv, CAR16-ScFv, CAR17-ScFv, CAR18-ScFv, CAR19-ScFv and CAR20-ScFv were subjected to homologous recombination PCR with CD8-CD28-CD3ζ to obtain complete ScFv-CD8-CD28-CD3ζ fragments, each of which was about 1600 bp. Enzyme digestion of vector plasmid and homologous recombination target fragments were carried out.

The purified CAR15-ScFv-CD8-CD28-CD3 ζ, CAR16-ScFv-CD8-CD28-CD3 ζ, CAR17-ScFv-CD8-CD28-CD3 ζ, CAR18-ScFv-CD8-CD28-CD3 ζ, CAR19-ScFv-CD8-CD28-CD3ζ, and CAR20-ScFv-CD8-CD28-CD3ζ were double-digested with XhoI and NotI restriction endonucleases, and the pMFG vector fragment was recovered by electrophoresis.

The target fragment was connected to the vector fragment, and the XhoI and NotI double-digested target fragments CAR15-ScFv-CD8-CD28-CD3 ζ, CAR16-ScFv-CD8-CD28-CD3ζ, CAR17-ScFv-CD8-CD28-CD3ζ, CAR18-ScFv-CD8-CD28-CD3ζ, CAR19-ScFv-CD8-CD28-CD3 ζ, and CAR20-ScFv-CD8-CD28-CD3 ζ recovered from the gel were connected to the pMFG vector enzyme-digested fragment, and the molar ratio of the target fragment to the vector fragment was 3:1-5:1.

The ligation product was transformed with DH5α competent cells, and then the plasmid DNA was extracted and identified by enzyme digestion. The extracted constructed plasmid DNA was double-digested with XhoI and NotI restriction endonucleases to identify whether the bands were correct, and the correct plasmid DNA was identified by enzyme digestion. The plasmid DNA that was correctly identified by enzyme digestion and purified was sent to Qingke Bio for sequencing. The sequencing primers are shown in the following table:

TABLE 2
Sequencing primers table

| Primer | |
|---|---|
| Name | Primer sequence |
| JW003 | GACACCAGACTAAGAACCTAGAAC (SEQ ID NO. 25) |
| JW004 | CTCAAAGTAGACGGCATCGCAGCT (SEQ ID NO. 26) |
| MFG-For | TAGACGGCATCGCAGCTTGGATAC (SEQ ID NO. 27) |
| MFG-Rev | CACCAGCTGAAGCCTATAGAGTAC (SEQ ID NO. 28) |

### II. Experimental Results

### 1. Schematic Diagram of BCMA CAR Structure

The ScFv region of BCMA CAR is a humanized monoclonal antibody sequence targeting BCMA. The SP gene and the myc gene for detecting expression efficiency are added before the ScFv gene sequence. The transmembrane region uses the CD8 hinge-transmembrane domain. The intracellular costimulatory molecule uses the CD28 molecule as the synergistic costimulatory domain, and the intracellular signal transduction molecule uses CD3ζ. The screened six BCMA CAR expression plasmid maps are shown in FIG. 2.

The results of gel electrophoresis of target fragment amplification showed that the gene synthesized pUC-BCMA CAR15, pUC-BCMA CAR16, pUC-BCMA CAR17, pUC-BCMA CAR18, pUC-BCMA CAR19, and pUC-BCMA CAR20 plasmids containing ScFv fragments were amplified by PCR, and the sizes were all approximately 800 bp; the plasmid containing the CD8-CD28-CD3ζ fragment was PCR amplified, and the fragment size was approximately 830 bp. The gel electrophoresis results showed that the sizes of the target bands were all correct.

### 2. Target Fragment Homologous Recombination Gel Electrophoresis Results

CAR15-ScFv, CAR16-ScFv, CAR17-ScFv, CAR18-ScFv, CAR19-ScFv, and CAR20-ScFv were recombined into CD8-CD28-CD3ζ by homologous recombination PCR. The size of the recombinant fragments was approximately 1.6 kb, and the gel electrophoresis showed that the band sizes were correct.

### 3. Gel Electrophoresis Results of Enzyme-Digested Vector Fragments

The plasmid containing the pMFG vector was double-digested with XhoI and NotI, and the fragment size was about 7 kb. The gel electrophoresis results showed that the fragment size was correct and could be used for the connection experiment with the target band.

### 4. Enzyme Digestion Identification Results

The constructed plasmid was double digested with XhoI and NotI for identification, and the fragment sizes were 7 kb and 1.6 kb. The gel electrophoresis results showed that the constructed pMFG-BCMA CAR15, pMFG-BCMA CAR16, pMFG-BCMA CAR17, pMFG-BCMA CAR18, pMFG-BCMA CAR19 and pMFG-BCMA CAR20 plasmid band sizes were correct and could be sent for sequencing for further verification.

### 5. Successful Construction of BCMA CAR Plasmid

The correct plasmids identified by enzyme digestion were purified and sent for sequencing. The sequencing results showed that the sequences of pMFG-BCMA CAR15, pMFG-BCMA CAR16, pMFG-BCMA CAR17, pMFG-BCMA CAR18, pMFG-BCMA CAR19 and pMFG-BCMA CAR20 were completely correct.

### Example 2. BCMA CAR Retroviral Vector Packaging

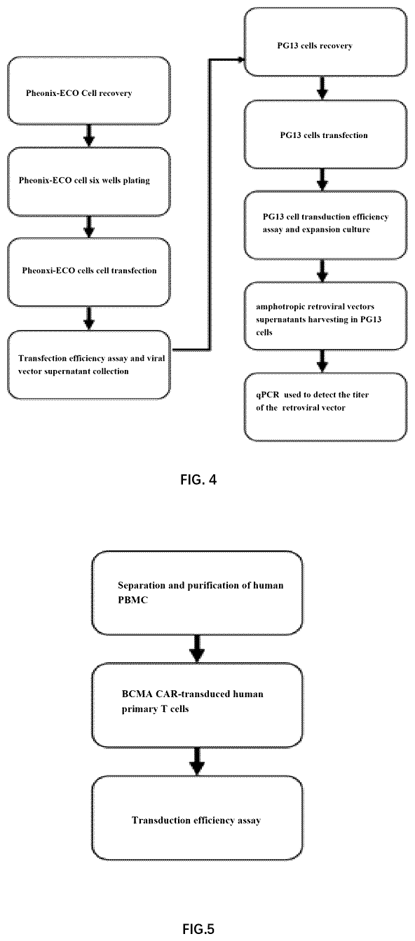

After the construction of the pMFG-BCMA CAR plasmid, the BCMA CAR retroviral vector was packaged to construct a stable production retroviral vector cell line. The specific experimental process is shown in FIG. 4: First, the pMFG-BCMA CAR plasmid was transiently transfected into Phoenix ECO cells to collect the BCMA CAR ecotropic retroviral vector supernatant particles. The collected BCMA CAR ecotropic retroviral vector particles were then transduced into the PG13 cell line for the production of dual-tropic retroviral vector supernatant particles, and a PG13 cell line that stably produced BCMA CAR dual-tropic retroviral vector supernatant particles was constructed. The viral vector titer was detected by qPCR to verify whether the BCMA CAR dual-tropic retroviral vector supernatant particles were successfully produced.

### I. Materials

The human eosinophilic packaging cell line Pheonix-ECO and the gibbon ape leukemia virus packaging cell line PG13 were purchased from the American type culture collection (ATCC).

DMEM complete medium: operating in a biosafety cabinet, adding 56 mL FBS (10%) and 5.6 mL penicillin-streptomycin (1%) to every 500 mL DMEM (89%); performing vacuum filtration on the prepared DMEM complete medium by using a 0.22 μm 500 mL filter; marking the bottle with reagent and store it in a 4° C. refrigerator for future use.

Cell freezing solution: FBS and DMSO are prepared according to the ratio of FBS:DMSO=9:1 in a biosafety cabinet; and the prepared cell freezing solution is filtered with a 0.22 μm filter membrane and backed up.

Staining buffer: Prepare PBS (1×) and FBS at a ratio of PBS:FBS=9:1 and store at 4° C. for later use.

### II. Experimental Methods

### 1. Phoenix ECO and PG13 Cell Culture

### 1.1. Cell Recovery

Take out the cells to be revived from the liquid nitrogen tank, resuspend the cell pellet with DMEM complete medium, transfer the cell suspension to an appropriate culture flask, and culture it in a 37° C., 5% CO₂ incubator.

### 1.2 Cell Passaging and Medium Replacement

Observe the cell adhesion and growth status under the microscope. If the cell density is less than 80%, but the culture medium turns yellow, the cell medium needs to be replaced. If the cell density is greater than 80%, the cell passage needs to be performed. Use an appropriate volume of 1×PBS to shake the culture bottle several times to wash the cells and wash the cells that have not successfully adhered. Gently discard the PBS used for washing, add an appropriate volume of trypsin to digest the cells, and gently shake the culture bottle to allow trypsin to fully contact the cells to accelerate cell digestion. It can also be placed in a 37° C. incubator to accelerate cell digestion. Observe whether the cells fall off under the microscope. When the cells fall off by about 90%, add three times the volume of trypsin to stop digestion. Transfer the cell suspension to a centrifuge tube and centrifuge at 300 g for 5 minutes. Carefully aspirate the supernatant with a pipette. Add an appropriate volume of new DMEM complete medium to resuspend the cell pellet. Transfer the cell suspension to an appropriate culture bottle and culture it in a 37° C., 5% CO₂ incubator. Observe the cell growth status under an inverted microscope every 24 hours.

### 1.3 Cell Cryopreservation

Add an appropriate volume of trypsin to digest the cells, digest at 37° C. for about 5 minutes, and take out the cells every 1 minute to observe the digestion status. When about 90% of the cells are digested and detached, add 3 times the volume of trypsin in DMEM complete medium to stop digestion. Use a pipette to gently blow the cell suspension into a single cell suspension, take out 20 μL of the single cell suspension and mix it with an equal volume of trypan blue solution. Add it dropwise to the cell counting plate and count it with a cell counter. Transfer the cell suspension to a centrifuge tube and centrifuge at 300 g for 5 minutes. Use a pipette to aspirate the supernatant and discard it. Be careful not to touch the cell pellet. Add cell freezing solution to resuspend the cells (the general cell freezing concentration is 5×10⁶-1×10⁷/mL). Add the cell freezing suspension to a 2 mL cryotube, put it into the program warm box, and immediately transfer it to a −80° C. refrigerator overnight.

### 2. Preparation of Ecotropic Retroviral Vector Supernatant

2.1 Plate Phoenix-ECO cells and discard the old culture medium of Phoenix-ECO cells that are growing well. Add trypsin to digest the cells and incubate at 37° C. for about 5 minutes to accelerate the digestion of the cells. Add 3 times the volume of trypsin in DMEM complete culture medium to terminate the digestion and centrifuge at 300 g for 5 minutes. Discard the liquid, add DMEM complete culture medium to resuspend the cells, take 20 μL of the single cell suspension and add it to an equal volume of trypan blue solution to count the cells.

Calculate and prepare the single cell suspension required for inoculation into a six-well cell culture plate at 1×10⁶ cells/well, 2.5 mL/well. Add the single cell suspension diluted to 1×10⁶ cells/well into the six-well culture plate. Gently shake the six-well culture plate back and forth or left and right to evenly distribute the cells. Place the six-well culture plate in a 37° C. incubator with 5% CO₂ and culture overnight.

### 2.2 Phoenix-ECO Cell Transfection

24 hours after cell inoculation, take out the six-well culture plate and observe under an inverted microscope. When the cells are growing well and the density reaches about 80%, transfection is performed. Calculate the required volume of plasmids pMFG-BCMA CAR15, pMFG-BCMA CAR16, pMFG-BCMA CAR17, pMFG-BCMA CAR18, pMFG-BCMA CAR19 and pMFG-BCMA CAR20, as well as the volume of Fugene HD.

Replace the six-well plate with 2.3 mL of DMEM complete medium per well. Gently drop 200 μL of the mixed solution into the six-well plate to make the total volume of each well 2.5 mL, and gently shake the plate. Place the six-well plate in a 37° C. incubator with 5% CO₂ for culture.

### 2.3 Harvesting of Ecotropic Retroviral Vector Supernatant

24 h after transfection, remove the six-well culture plate from the incubator, carefully remove the culture supernatant, slowly add new DMEM complete medium, 2.5 mL/well; move the six-well culture plate to a 32° C. incubator containing 5% CO₂ for culture. 48 h after transfection, carefully collect the supernatant into a centrifuge tube; slowly add new DMEM complete medium, 2.5 mL/well; move the six-well culture plate to a 32° C., 5% CO₂ incubator for culture; the collected culture supernatant is filtered with a 0.45 μm low adsorption virus filter membrane, and stored in aliquots at −80° C. for use. 72 h after transfection, remove the six-well culture plate and place it in a biosafety cabinet; carefully collect the supernatant into a centrifuge tube; the collected culture supernatant is filtered with a 0.45 μm low adsorption virus filter membrane, and stored in aliquots at −80° C. for use.

### 2.4 Phoenix-ECO Cell Transfection Efficiency Detection

72 h after the above transfection, Phoenix-ECO cells were digested with trypsin and the cell suspension was collected after termination. Centrifuge at 300 g for 5 min, discard the liquid, collect the cell pellet, and wash the cells with PBS. Centrifuge at 300 g for 5 min at room temperature and discard the supernatant. Resuspend the cells with 50 L staining buffer, add anti-hc-Myc PE antibody for staining, and use untransfected Phoenix-ECO cells as negative control. After staining at 4° C. in the dark for 1 h, add 900 L PBS to wash the cells. Centrifuge at 300 g for 5 min, discard the supernatant, and resuspend the cell pellet with staining buffer. Detect transfection efficiency by flow cytometry.

### 3. Preparation of Amphotropic Retroviral Vectors

3.1. PG13 cell transduction, pre-treat non-treated 12-well culture plates with RetroNectin 1 day before transduction, and dilute RetroNectin with PBS to a final concentration of 10 μg/mL, 1 mL per well. Grouping: PG13 cell control group, BCMA CAR15, BCMA CAR16, BCMA CAR17, BCMA CAR18, BCMA CAR19 and BCMA CAR20 experimental groups. The supernatant of the ecotropic retroviral vector collected 72 h after transfection with Phoenix-ECO was taken out from −80° C., re-thawed at room temperature, and 1 mL was added to each well of the 12-well culture plate. The 12-well culture plate with the ecotropic retroviral vector supernatant was centrifuged at 30° C. for 1 h. Take out the PG13 cells from the 37° C. incubator, observe the cell state under a microscope, trypsinize and terminate, and pipette into a single cell suspension. Count the cells, take 1× 10⁶ cells from each well, transfer them to a centrifuge tube, and centrifuge at 300 g for 5 minutes.

48 hours after transduction, a portion of the cells were digested and taken for transduction efficiency detection, and the remaining PG13 cells were subcultured and expanded to T 75 culture flasks. When the cell density reached 80%, the dual-tropic retroviral vector supernatant was harvested and named H0, and new DMEM complete medium was added, and the culture plate was transferred to a 32° C., 5% CO₂ incubator for culture, and the viral vector supernatant was pulled out and stored at −80° C. After that, the viral vector supernatant was harvested for 4 consecutive days and named H1-H4. The harvested viral vector supernatant was stored at −80° C. for standby use. PG13 cell transduction efficiency detection, 48 hours after transduction, a small amount of PG13 cells was taken to detect the transduction efficiency. qPCR was used to detect the titer of the BCMA CAR retroviral vector, and the viral titer of the BCMA CAR dual-tropic retroviral vector supernatant produced by the above-collected PG13 cell line was detected to verify whether the BCMA CAR dual-tropic retroviral vector particles were successfully prepared.

### III. Experimental Results

### 1. Successfully Prepared BCMA CAR Ecotropic Retroviral Vector

Phoenix-ECO cells were transfected with pMFG-BCMA CAR15, pMFG-BCMA CAR16, pMFG-BCMA CAR17, pMFG-BCMA CAR18, pMFG-BCMA CAR19 and pMFG-BCMA CAR20 plasmids. The transfection efficiency was detected by flow cytometry 72 hours later. The transfection efficiency of pMFG-BCMA CAR15 was 56.70%, pMFG-BCMA CAR16 was 64.91%, pMFG-BCMA CAR17 was 59.11%, pMFG-BCMA CAR18 was 57.88%, pMFG-BCMA CAR19 was 59.85%, and pMFG-BCMA CAR20 was 57.95%. All six plasmids were successfully transfected in Phoenix-ECO cells. The supernatant of the ecotropic retroviral vector collected 72 hours after transfection can be used to transduce PG13 cells.

2. Successfully Constructed a Cell Line that Stably Produces BCMA CAR Dual-Tropic Retroviral Vector

The successfully prepared BCMA CAR15, BCMA CAR16, BCMA CAR17, BCMA CAR18, BCMA CAR19 and BCMA CAR20 ecotropic retroviral vector supernatants were transduced into PG13 cells, and the transduction efficiency was detected by flow cytometry 48 hours later. The transduction efficiency of BCMA CAR15 was 96.91%, the transduction efficiency of BCMA CAR16 was 97.53%, the transduction efficiency of BCMA CAR17 was 98.50%, the transduction efficiency of BCMA CAR18 was 98.69%, the transduction efficiency of BCMA CAR19 was 97.82%, and the transduction efficiency of BCMA CAR20 was 98.62%. The results showed that the amphotropic retroviral vector particles were successfully prepared, and the PG13 cell line that stably produced amphotropic retroviral vector particles was successfully constructed.

### 3. Successful Preparation of BCMA CAR Dual-Tropic Retroviral Vector

The PG13 cell line transduced with BCMA CAR ecotropic retroviral vector was expanded and cultured in T75 culture flasks, and the amphotropic retroviral vector particles were harvested for 5 consecutive days for qPCR detection. The results showed that the highest titers of BCMA CAR amphotropic retroviral vectors were BCMA CAR15H2: 1.40×10⁷±1.13×10⁶ copies/mL, BCMA CAR16H1: 1.33×10⁷+7.07×10⁴ copies/mL, BCMA CAR17H2: 3.09×10⁷±1.70×10⁶ copies/mL, BCMA CAR18H2: 2.44×10⁷±2.55×10⁶ copies/mL, BCMA CAR19H2: 1.95×10⁷±2.36×10⁴ copies/mL, BCMA CAR20H2: 2.68×10⁷±4.24×10⁶ copies/mL, both higher than the positive control. This result shows that we have successfully prepared BCMA CAR15, BCMA CAR16, BCMA CAR17, BCMA CAR18, BCMA CAR19, and BCMA CAR20 dual-tropic retroviral vectors, and the dual-tropic retroviral vector particles with the highest titer for each BCMA CAR were selected for human primary T cell transduction experiments.

### Example 3 Preparation of BCMA CAR-T Cells

On the basis of preparing BCMA CAR dual-tropic retroviral vector particles, BCMA CAR dual-tropic retroviral vector particles are transduced into human primary T cells to construct BCMA CAR-T cells. The technical route is shown in FIG. 5: First, human peripheral blood PBMCs are separated by density gradient centrifugation, primary T cells are activated under the stimulation of CD3 monoclonal antibody and cytokine IL-2, and activated T cells are transduced with BCMA CAR by centrifugation to construct BCMA CAR-T cells. The expression of Myc tag on the cell surface is detected by flow cytometry to detect whether BCMA CAR is expressed on the surface of T cells.

### I. Experimental Methods

### 1. Construction of BCMA CAR-T Cells

One day before transduction, non-treated 12-well culture plates were pretreated with RetroNectin. RetroNectin was diluted with PBS to a final concentration of 10 μg/mL, and 1 mL was added to each well.

Grouping: T cell non-transduced control group (Pan-T group), BCMA CAR15-T group, BCMA CAR16-T group, BCMA CAR17-T group, BCMA CAR18-T group, BCMA CAR19-T group, BCMA CAR20-T group and bb2121 CAR-T (currently available BCMA CAR, except for the ScFv, the rest of the structure is the same as other BCMA CARs we constructed) group. The experiment was repeated by transducing T cells isolated from different volunteers.

Aspirate the RetroNectin solution, add 1 mL of PBS to the 12-well culture plate for washing, and discard the solution. Take out the supernatant of the amphotropic retroviral vector collected from the PG13 cell line from −80° C., rethaw at room temperature, and add 1 mL to each well of the 12-well culture plate. Centrifuge the 12-well culture plate with the amphotropic retroviral vector supernatant at 30° C. for 1 hour. Take out the T cells from the 37° C. incubator, observe the cell state under a microscope, and pipette into a single cell suspension. Count the cells, take 1×10⁶ cell suspension from each well, transfer to a centrifuge tube, and centrifuge at 300 g for 5 minutes. Discard the solution, resuspend the cells in 1 mL of the amphotropic retroviral vector supernatant in each well, and resuspend the control group in AIM-V complete medium. Blow the cells into a single cell suspension and slowly add it dropwise to the 12-well culture plate. Centrifuge the 12-well culture plate with the cells resuspended in the amphotropic retroviral vector supernatant at 30° C. for 1 hour. Place the 12-well culture plate in a 37° C., 5% CO₂ incubator and culture for at least 1 hour. Take out the 12-well culture plate, digest and collect the cells, centrifuge at 300 g for 5 minutes, and carefully remove the supernatant. Slowly add new amphotropic retroviral vector supernatant, 1 mL per well, and add new AIM-V complete medium to the control group. Collect the cells, centrifuge at 300 g for 5 minutes, and carefully remove the supernatant. Resuspend the cells with fresh AIM-V complete medium. Place the 12-well culture plate in a 37° C., 5% CO₂ incubator and culture.

### 2. T Cell Transduction Efficiency Detection

After 48 hours of transduction, human primary T cells were mixed with a pipette and gently blown into a single cell suspension. Take 300 μL of cell suspension into a 1.5 mL centrifuge tube. Centrifuge at 300 g for 5 minutes, collect the cell pellet, and wash the cells with PBS. Centrifuge at 300 g for 5 minutes at room temperature and discard the supernatant. Resuspend the cells with 50 μL staining buffer, add APC anti-human CD3 antibody and anti-hc-Myc PE antibody for staining, and use untransduced T cells as negative control. After staining at 4° C. in the dark for 1 hour, add 900 μL PBS to wash the cells. Centrifuge at 300 g for 5 minutes, discard the supernatant, and resuspend the cell pellet with staining buffer. Use flow cytometry to detect the transduction efficiency. The percentage of CD3-positive and Myc-positive cells is the transduction efficiency of BCMA CAR transduced T cells.

### II. Experimental Results

BCMA CAR retroviral vector successfully transduced human primary T cells. The BCMA CAR dual-tropic retroviral vector was transduced into human primary T cells, and the transduction efficiency was detected 48 hours later. The transduction efficiency of bb2121 CAR-T was 55.30%, the transduction efficiency of BCMA CAR15-T was 43.18%, the transduction efficiency of BCMA CAR16-T was 55.35%, the transduction efficiency of BCMA CAR17-T was 65.87%, the transduction efficiency of BCMA CAR18-T was 62.66%, the transduction efficiency of BCMA CAR19-T was 55.42%, and the transduction efficiency of BCMA CAR20-T was 54.59%. The results of this study showed that we successfully constructed BCMA CAR-T cells, which can be used in subsequent experiments to verify their ability to kill tumor cells.

### Example 4 Verification of BCMA CAR-T Anti-Tumor Function In Vitro

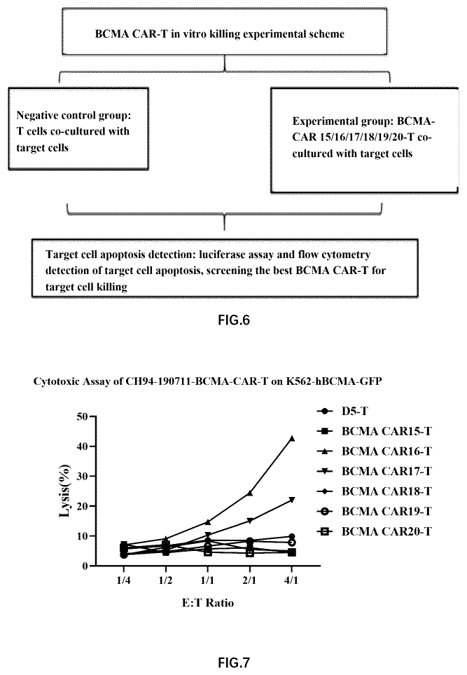

On the basis of the successful construction of BCMA CAR-T cells, its ability to kill tumor cells was verified. BCMA CAR-T cells were co-incubated with target cells, and the apoptosis of target cells was detected by luciferase flow cytometry to verify the killing ability of BCMA CAR-T cells on tumor cells. The technical route is shown in FIG. 6.

### I. Luciferase Bioluminescence Assay to Detect the Anti-Tumor Ability of BCMA CAR-T In Vitro

Firefly luciferase is a monomeric protein with a size of about 61 kDa. The substrate ATP-Mg²⁺ can catalyze the oxidation of luciferin. The chemical energy in the oxidation process is converted into electron transition to generate light energy, forming the product molecule oxidized luciferin. The RPMI-gfp-luc cells stably expressing the luciferase reporter gene stored in the laboratory can generate chemical signals under the catalytic action of the substrate to detect the survival of tumor cells.
1. Grouping: Pan-T group, BCMA CAR15-T group, BCMA CAR16-T group, BCMA CAR17-T group, BCMA CAR18-T group, BCMA CAR19-T group and BCMA CAR20-T group.
2. Plating: Blow RPMI-gfp-luc into a single cell suspension and count the cells; when the cells are in good growth condition, dilute the cells to 4×10^{4/50} μL, take 50 μL/well, and inoculate them into a 96-well all-white culture plate.
3. Take 50 μL/well of Pan-T, BCMA CAR15-T, BCMA CAR16-T, BCMA CAR17-T, BCMA CAR18-T, BCMA CAR19-T and BCMA CAR20-T cells and mix them with target cells at different effector-target ratios (1:4, 1:2, 1:1, 2:1, 4:1) (the tumor cell group was set as a blank control). Culture in a 37° C., 5% CO₂ incubator for 12 h.
4. Add ONE-Glo™ Luciferase Assay Reagent in an equal volume to the culture medium to each well and mix thoroughly.
5. The following formula was used for analysis: Cell lysis rate=1-(lysis of experimental group−lysis of blank group)/(lysis of maximum release pore-lysis of blank group)×100%. Each experiment was repeated three times.
6. Data processing: GraphPad Prism 8 software was used for statistical analysis. The quantitative data were expressed as (x±s). The t-test was used for comparison between the two groups. When P<0.05, the difference was considered statistically significant.

### II. Flow Cytometry to Detect the Anti-Tumor Ability of BCMA CAR-T In Vitro

Annexin V is a phospholipid-binding protein that has a high affinity for phosphatidylserine (PS). It can specifically bind to the cell membrane of early apoptotic cells through PS exposed on the extracellular side of the cells. Therefore, Annexin V is a sensitive indicator for detecting early cell apoptosis.
1. Grouping: Pan-T group, BCMA CAR15-T group, BCMA CAR16-T group, BCMA CAR17-T group, BCMA CAR18-T group, BCMA CAR19-T group and BCMA CAR20-T group.
2. Plating: Blow K562-hBCMA-gfp into a single cell suspension and count the cells; when the cells are in good growth condition, dilute the cells to 4×10⁴/100 μL, take 100 μL/well, and inoculate them in a 96-well plate.
3. The effector cells were mixed with target cells at different effector-target ratios (1:4, 1:2, 1:1, 2:1, 4:1) (the tumor cell group was set as a blank control), and cultured in a 37° C., 5% CO₂ incubator for 12 h.
4. Add 130 μL staining buffer to each well to wash the cells, centrifuge at 300 g for 5 min, and discard the supernatant.
5. Add BV421 anti-human CD3 antibody to each well and stain for 40 min at 4° C. in the dark.
6. Add 150 μL staining buffer to each well to terminate staining, centrifuge at 300 g for 5 min, and discard the supernatant.
7. Add Annexin V-Alexa Fluor 647 to each well to detect tumor cell apoptosis and stain for 40 minutes at 4° C. in the dark.
8. Add 150 μL staining buffer to each well to terminate staining, centrifuge at 300 g for 5 min, and discard the supernatant.
9. Resuspend cells in 200 μL staining buffer per well.
10. Data were collected by flow cytometry and analyzed by FlowJo. The apoptosis rate of tumor cells was calculated as the percentage of CD3-negative and Annexin V-positive cells in total cells. The experiment was repeated three times by sampling blood from different volunteers. The BCMA CAR-T with the best killing effect was screened out.

### III. Flow Cytometry Further Verifies the Anti-Tumor Ability of BCMA CAR-T in Vitro

1. Grouping: Pan-T group, positive control group (bb2121 CAR-T group), and BCMA CAR16-T group.
2. Plating: Blow RPMI-gfp-luc into a single cell suspension and count the cells; when the cells are in good growth condition, dilute the cells to 4×10⁴/100 μL, take 100 μL/well, and inoculate them in a 96-well plate.
3. Pan-T, bb2121 CAR-T, and BCMA CAR16-T cells were mixed with target cells at different effector-target ratios (1:4, 1:2, 1:1, and 2:1) (the tumor cell group was set as a blank control) and cultured in a 37° C., 5% CO₂ incubator for 12 h.
4. The flow cytometry method and data processing are the same as the above steps.

### IV. Experimental RESULTS

### 1. Detection Results of BCMA Expression on the Surface of Tumor Cells

K562-hBCMA-gfp, RPMI-gfp-luc, K562-cBCMA, and K562 tumor cells were stained with BV421 anti-human BCMA antibody, and the expression of surface BCMA was detected by flow cytometry. The results showed that BCMA was highly expressed on the surface of K562-hBCMA-gfp and RPMI-gfp-luc cells to varying degrees, among which the expression of BCMA on the surface of K562-hBCMA-gfp was 85.5%, and the expression of BCMA on the surface of RPMI-gfp-luc was 58.5%, so they can be used as target cells in this study; K562-cBCMA and K562 cells basically do not express BCMA on their surfaces and can be used as negative control cells.

### 2. In Vitro Anti-Tumor Activity of BCMA CAR-T Cells Detected by Luciferase Bioluminescence Assay

In order to verify the killing ability of BCMA CAR-T on tumor cells in vitro, we mixed BCMA CAR15-T, BCMA CAR16-T, BCMA CAR17-T, BCMA CAR18-T, BCMA CAR19-T and BCMA CAR20-T cells with target cells at different effector-target ratios. After 12 hours, the apoptosis rate of target cells was detected by luciferase bioluminescence. The results showed that compared with the Pan-T group, the killing ability of BCMA CAR16-T group and BCMA CAR17-T group on tumor cells was enhanced, and it was positively correlated with the effector-target ratio. BCMA CAR16-T showed the strongest killing effect on tumor cells. Three independent repeated experiments showed the same results.

### 3. In Vitro Tumor Killing Activity of BCMA CAR-T Cells Detected by Flow Cytometry

In order to repeatedly verify the killing ability of BCMA CAR-T on tumor cells in vitro, we mixed BCMA CAR15-T, BCMA CAR16-T, BCMA CAR17-T, BCMA CAR18-T, BCMA CAR19-T and BCMA CAR20-T cells with target cells K562-hBCMA-gfp at different effector-target ratios. After 12 hours, the apoptosis rate of target cells was detected by flow cytometry. The results are shown in the figure: Compared with the Pan-T group, the killing ability of BCMA CAR16-T group and BCMA CAR17-T group on tumor cells was enhanced, and this killing ability was positively correlated with the effector-target ratio. BCMA CAR16-T showed the strongest killing effect on tumor cells. Three independent repeated experiments showed the same results, which were consistent with the results of firefly bioluminescence detection, as shown in FIG. 7. The BCMA CAR16-T with the best killing effect was selected and compared with the positive BCMA CAR (bb2121 CAR-T).

### 4. Further Verification of BCMA CAR-T's Anti-Tumor Ability In Vitro

The BCMA CAR16-T with the most significant killing effect on tumor cells was screened out and the killing effect was further compared with the positive bb2121 CAR-T cells. The results are shown in FIG. 8 below: Although the killing effect of BCMA CAR16-T on tumor cells is more significant than that of the Pan-T group, it is not as good as that of the positive control group bb2121 CAR0-T, and the difference is statistically significant. Based on the screened BCMA CAR16, further optimization is needed to improve the killing efficiency of tumor cells.

### Example 5 Optimization of BCMA CAR Retroviral Vector Plasmid Construction and BCMA CAR Retroviral Vector Packaging

### I. Optimization of BCMA CAR Retroviral Vector Plasmid Construction

We have successfully screened out the BCMA CAR16-T cells with the strongest ability to kill tumor cells. However, compared with the bb2121 CAR-T cells that have been on the market, the ability to kill tumor cells needs to be enhanced. Since the structures of BCMA CAR16-T and bb2121 CAR-T are exactly the same except for the ScFv, we designed to connect two or three BCMA CAR16-T ScFvs in series to improve its antigen affinity, in order to enhance the anti-tumor ability.

### 1. Schematic Diagram of the Structure of BCMA CAR31, BCMA CAR32, and BCMA CAR33

The ScFv region of BCMA CAR uses the humanized BCMA-targeting monoclonal antibody sequence BCMA CAR16 screened by phage display in the previous laboratory. The three optimized BCMA CAR structure ScFv regions are two SvFvs connected and three ScFv regions connected, and the rest of the structure remains unchanged. The newly constructed BCMA CAR expression plasmid map is shown in FIG. 9.

2. Gel electrophoresis results of target fragment and vector fragment

After double digestion with XhoI and NgoMIV, the size of the vector pMFG fragment was about 7 kb, the size of the target fragment BCMA CAR31 was about 1.7 kb, the size of the BCMA CAR32 fragment was about 1.7 kb, and the size of the BCMA CAR33 fragment was about 2.5 kb. The electrophoresis results showed that the sizes of the vector fragment and the target fragment bands were correct.

3. pMFG-BCMA CAR Enzyme Digestion and Gel Electrophoresis Results

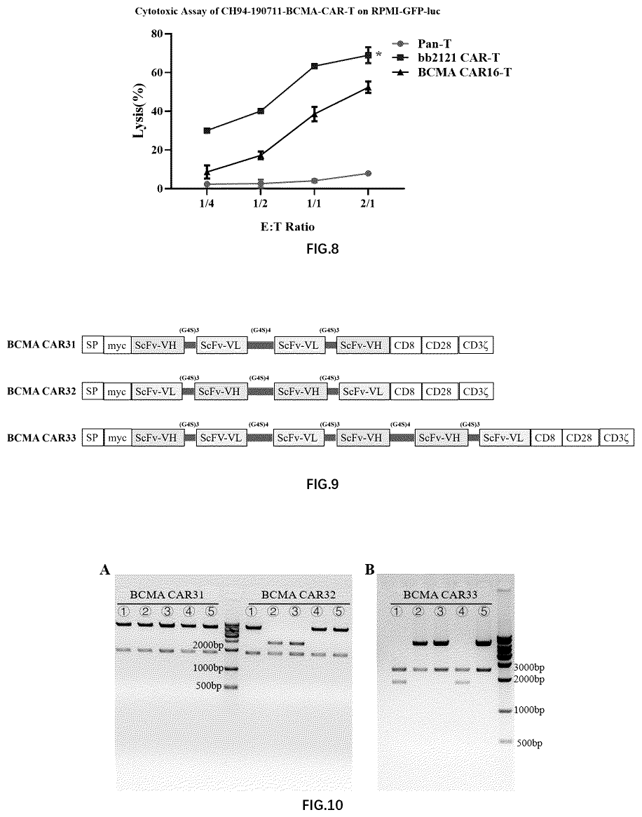

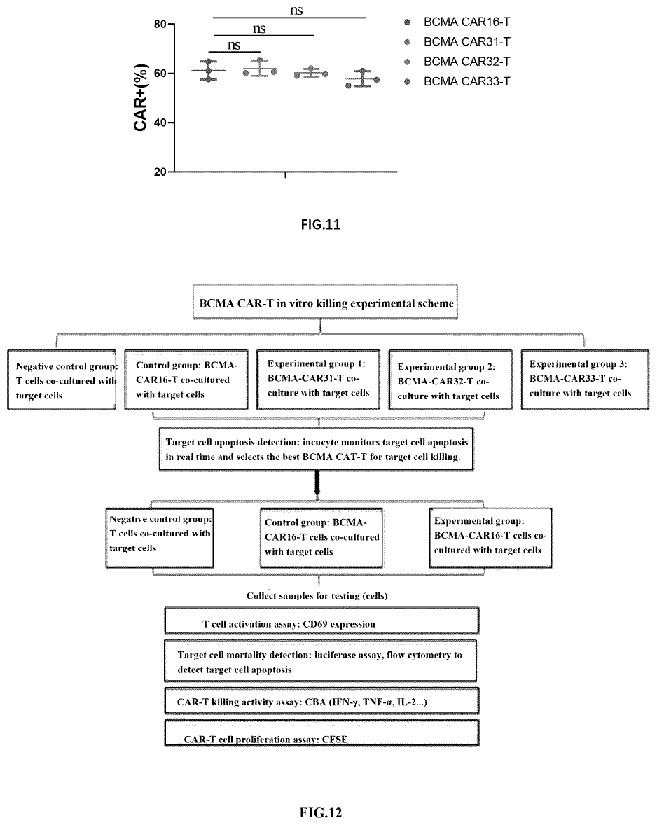

The constructed pMFG-BCMA CAR31, pMFG-BCMA CAR32, and pMFG-BCMA CAR33 plasmids were extracted and identified by double enzyme digestion (XhoI/NgoMIV). The target fragment BCMA CAR31 fragment size was about 1.7 kb, the BCMA CAR32 fragment size was about 1.7 kb, the BCMA CAR33 fragment size was about 2.5 kb, and the vector pMFG fragment size was about 7 kb. The electrophoresis results are shown in FIG. 10: pMFG-BCMA CAR31 plasmid enzyme digestion identification \textcircled{1}\textcircled{2}\textcircled{3}\textcircled{4}\textcircled{5} were all correct; pMFG-BCMA CAR32 plasmid enzyme digestion identification \textcircled{1}4\textcircled{ }\textcircled{5} were correct; pMFG-BCMA CAR33 plasmid enzyme digestion identification \textcircled{2}\textcircled{3}\textcircled{5} were correct. The correctly identified plasmid was sent for sequencing for further identification. The plasmid that was correctly identified by enzyme digestion was purified and sent for sequencing. The sequencing results showed that the sequences of pMFG-BCMA CAR31, pMFG-BCMA CAR32, and pMFG-BCMA CAR33 were completely correct.

### II. Optimizing BCMA CAR Retroviral Vector Packaging

On the basis of the optimized pMFG-BCMA CAR plasmid, the BCMA CAR retroviral vector was packaged to construct a stable production retroviral vector cell line. The specific experimental process is shown in FIG. 4: First, the optimized pMFG-BCMA CAR plasmid was transiently transfected into Phoenix ECO cells to collect the BCMA CAR ecotropic retroviral vector supernatant particles. The collected BCMA CAR ecotropic retroviral vector supernatant particles were then transduced into the PG13 cell line for the production of dual-tropic retroviral vector supernatant particles, and a PG13 cell line that stably produced BCMA CAR dual-tropic retroviral vector supernatant particles was constructed. The viral vector titer was detected by qPCR to verify whether the BCMA CAR dual-tropic retroviral vector supernatant particles were successfully produced. Using a similar method as before, the following results were obtained.

### 1. Successfully Prepared BCMA CAR Ecotropic Retroviral Vector

The pMFG-BCMA CAR31, pMFG-BCMA CAR32, and pMFG-BCMA CAR33 plasmids were transfected into Phoenix-ECO cells, and the transfection efficiency was detected by flow cytometry 72 hours later: the transfection efficiency of pMFG-BCMA CAR31 was 59%, the transfection efficiency of pMFG-BCMA CAR32 was 58.07%, and the transfection efficiency of pMFG-BCMA CAR33 was 60.62%. All three plasmids were successfully transfected into Phoenix-ECO cells.

2. Successfully Constructed a Cell Line that Stably Produces BCMA CAR Dual-Tropic Retroviral Vector

The successfully prepared BCMA CAR31, BCMA CAR32, and BCMA CAR33 ecotropic retroviral vector supernatants were transduced into PG13 cells, and the transduction efficiency was detected by flow cytometry 48 hours later. The results showed that the transduction efficiency of BCMA CAR31 was 93.66%, the transduction efficiency of BCMA CAR32 was 95.35%, and the transduction efficiency of BCMA CAR33 was 98.24%. The results showed that the amphibious retroviral vector particles were successfully prepared, and the PG13 cell line that stably produced amphibious retroviral vector particles was successfully constructed.

### 3. Successful Preparation of BCMA CAR Dual-Tropic Retroviral Vector

The PG13 cell lines transduced with BCMA CAR31, BCMA CAR32 and BCMA CAR33 ecotropic retroviral vectors were expanded and cultured in T75 culture flasks, and the amphotropic retroviral vector particles were harvested for 5 consecutive days for qPCR detection. The results showed that the highest titer of BCMA CAR31 retroviral vector was BCMA CAR31 H4: 2.29×10⁷±1.06×10⁶ copies/mL, BCMA CAR32 H3: 1.64×10⁷±1.12×10⁶ copies/mL, and BCMA CAR33 H4: 2.40×10⁷±4.53×10⁶ copies/mL, all of which were higher than the positive control. This result shows that we have successfully prepared BCMA CAR31, BCMA CAR32, and BCMA CAR33 amphotropic retroviral vectors, and selected the amphotropic retroviral vector particles with the highest titer for transduction of primary human T cells.

### Example 6 Optimization of BCMA CAR-T Cell Preparation

On the basis of the successful preparation of BCMA CAR dual-tropic retroviral vector particles, BCMA CAR dual-tropic retroviral vector particles were transduced into human primary T cells to construct BCMA CAR-T cells. The technical route is shown in FIG. 5: First, human peripheral blood PBMCs were separated by density gradient centrifugation, primary T cells were activated under the stimulation of CD3 monoclonal antibody and cytokine IL-2, and the activated T cells were transduced with BCMA CAR by centrifugation to construct BCMA CAR-T cells. The expression of Myc tag on the cell surface was detected by flow cytometry to detect whether BCMA CAR was expressed on the surface of T cells; further, the copy number of BCMA CAR retroviral vector in each primary T cell genome was detected by qPCR to detect whether it was successfully integrated into the human primary T cell genome. Using a similar method as before, the following results were obtained.

### I. BCMA CAR Retroviral Vector Successfully Transduced Human Primary T Cells

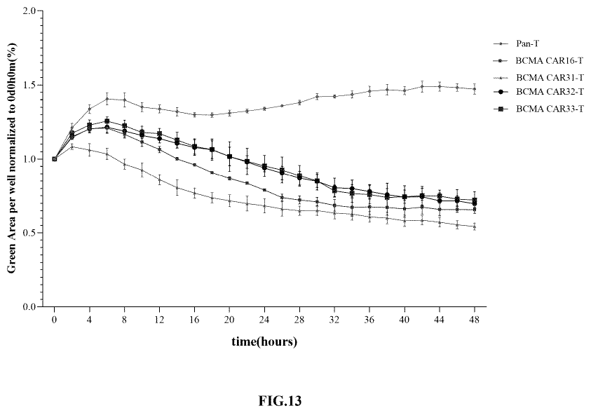

The BCMA CAR amphotropic retroviral vector was transduced into human primary T cells, and the transduction efficiency was detected after 48 hours. The results are shown in FIG. 11: the transduction efficiency of BCMA CAR16-T was 61.10%±3.65%, the transduction efficiency of BCMA CAR31-T was 61.97%±2.98%, the transduction efficiency of BCMA CAR32-T was 62.20%±1.59%, and the transduction efficiency of BCMA CAR33-T was 57.80%±3.02%. The results showed that the transduced human primary T cells successfully expressed BCMA CAR, and there was no statistical difference in the transduction efficiency between the three groups compared with the control group BCMA CAR16-T. We successfully prepared BCMA CAR-T cells. BCMA CAR successfully integrated into the human primary T cell genome. The BCMA CAR dual-tropic retroviral vector was used to transduce human primary T cells, and the number of copies integrated into the T cell genome was detected 48 hours later. The results showed that the number of copies of BCMA CAR16-T integrated into the genome was 0.44±0.05 copies/T cell, the number of copies of BCMA CAR31-T integrated into the genome was 0.48±0.02 copies/cell, the number of copies of BCMA CAR32-T integrated into the genome was 0.43±0.04 copies/T cell, and the number of copies of BCMA CAR33-T integrated into the genome was 0.32±0.04 copies/T cell. The results showed that BCMA CAR was successfully integrated into the human primary T cell genome.

### Example 7 Optimization of BCMA CAR-T In Vitro Anti-Tumor Function Verification

On the basis of successfully constructing three optimized BCMA CAR-T cells, their killing function against tumor cells in vitro was first verified. Using different tumor cells expressing human BCMA antigen as target cells and target cells that do not express human BCMA antigen as controls, the survival of tumor cells was monitored by luciferase bioluminescence experiment and incucyte real-time dynamic live cell imaging, and the apoptosis of tumor cells was detected by flow cytometry to verify the killing specificity and effectiveness of BCMA CAR-T cells on target cells; the level of cytokine secretion by BCMA CAR-T cells was detected by CBA experiment; and the proliferation ability of BCMA CAR-T was detected by CFSE experiment. The specific experimental scheme is shown in FIG. 12. Using similar methods as before, the following experimental results were obtained.

### I. BCMA CAR31-T has Significant Killing Ability In Vitro

BCMA CAR16-T, BCMA CAR31-T, BCMA CAR32-T, and BCMA CAR33-T were co-incubated with K562-hBCMA-gfp cells at an effector-target ratio of 1:1, and the fluorescence changes of tumor cells were continuously monitored by real-time fluorescence, recorded every 2 hours, and continuously monitored for 48 hours. The results are shown in FIG. 13: BCMA CAR31-T has a stronger killing effect on tumor cells than the control group BCMA CAR16-T. Therefore, in the next experiment, we selected BCMA CAR31-T to continue to compare with BCMA CAR16-T for tumor killing, cytokine secretion and proliferation ability.

### 1. BCMA CAR31-T can Effectively Activate

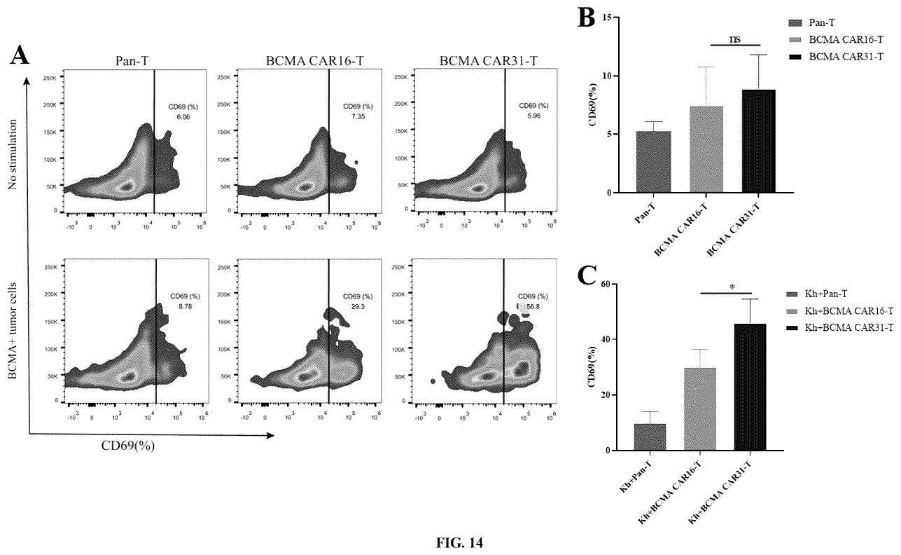

Flow cytometry was used to detect the expression of CD69 on the surface of BCMA CAR-T cells. The results are shown in FIG. 14: in the absence of tumor cell stimulation, there was no statistical difference in the expression of surface CD69 between BCMA CAR31-T and BCMA CAR16-T. Under the stimulation of K562-hBCMA-gfp cells expressing BCMA, the expression of surface CD69 of BCMA CAR31-T cells was higher than that of BCMA CAR16-T cells, and the difference was statistically significant. This study result shows that the optimized BCMA CAR31-T can be better activated by tumor cells.

### 2. BCMA CAR31-T has Highly Effective Anti-Tumor Ability In Vitro

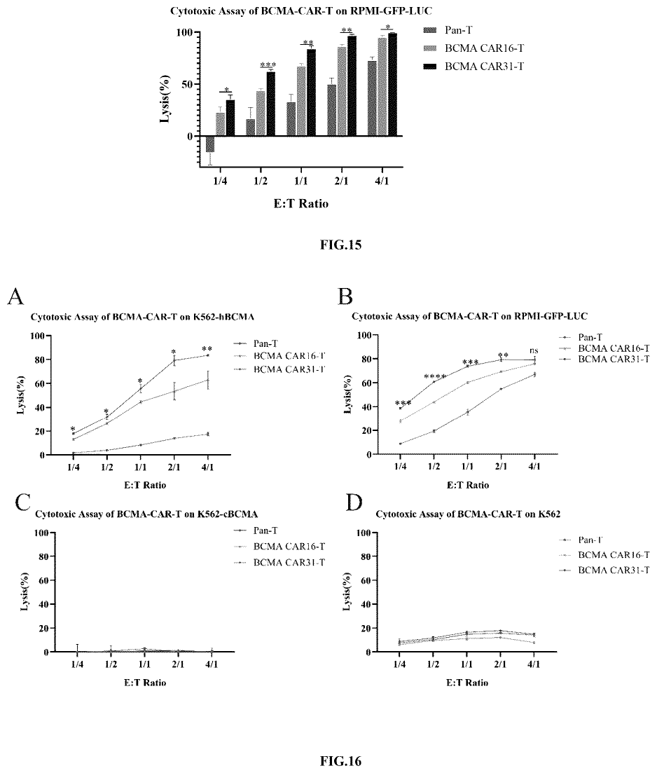

In order to determine the lysis ability of BCMA CAR31-T cells on BCMA-positive tumor cells, BCMA CAR31-T cells, BCMA CAR16-T cells, or Pan-T were incubated with RPMI-gfp-luc or cells at different effector-target ratios for 12 h, and the chemiluminescent signal intensity was detected. The experimental results are shown in FIG. 15: compared with BCMA CAR16-T, BCMA CAR31-T showed a stronger ability to kill tumor cells in vitro at each effector-target ratio, and the difference was statistically significant.

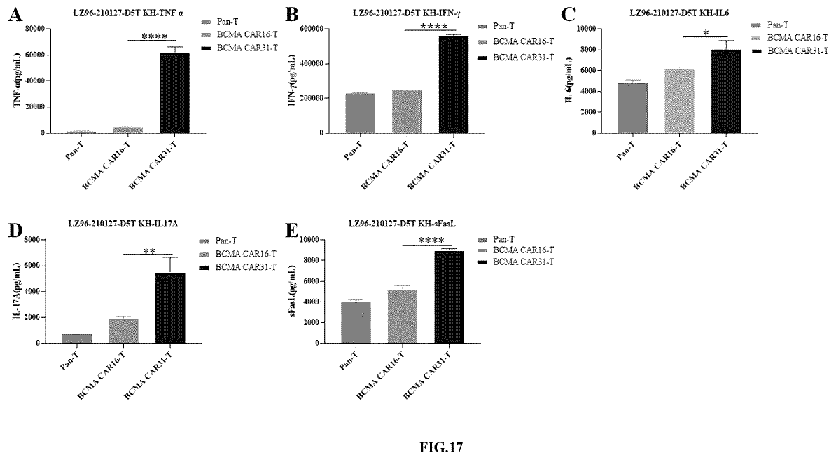

BCMA CAR31-T has efficient and specific anti-tumor ability in vitro. In order to verify the antigen specificity of the anti-tumor effect of BCMA CAR31-T cells, BCMA CAR31-T cells were co-cultured with K562-hBCMA-gfp and PMI-gfp-luc cells expressing human BCMA antigen at different effector-target ratios. K562-cBCMA expressing cynomolgus monkey BCMA and K562 cells not expressing BCMA were used as negative target cell controls. BCMA CAR16-T control group. The samples were detected by flow cytometry. The results are shown in FIG. 16. Compared with the BCMA CAR16-T group, BCMA CAR31-T cells have stronger killing power against two different BCMA-positive tumor cells at different effector-target ratios. However, BCMA CAR31-T showed the same killing ability as Pan-T for non-human BCMA-expressing K562-cBCMA and BCMA-negative K562 cells. This result shows that BCMA CAR31-T cells are specific for the BCMA antigen of tumor cells.

### 3. BCMA CAR31-T has Strong Cytokine Secretion Ability

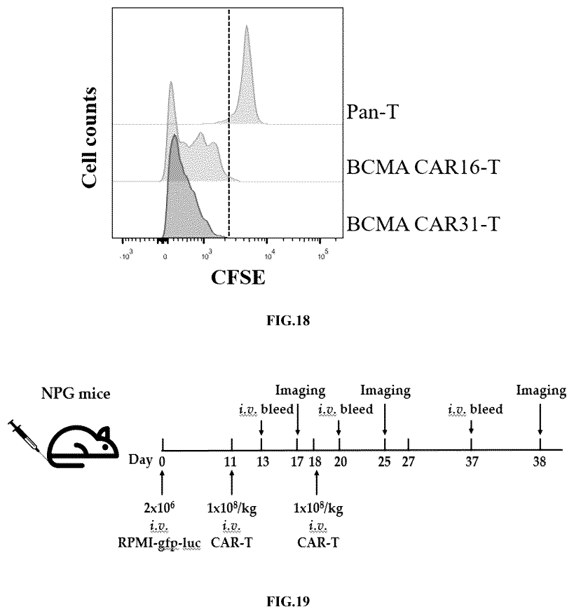

The production of cytokines is a sign that CAR-T cells are effectively activated. BCMA CAR31-T cells were co-incubated with K562-hBCMA-gfp cells expressing human BCMA antigen at a 1:1 effector-target ratio for 12 h. The cell culture fluid was harvested and cytokines such as TNF-α, IFN-γ, IL-6, IL-17A, and aFasL were measured using a CBA kit. The results are shown in FIG. 17: compared with BCMA CAR16-T cells, BCMA CAR31-T cells secreted significantly more cytokines TNF-«, IFN-γ, IL-6, IL-17A, and aFasL under the stimulation of BCMA-positive tumor cells. The release of these cytokines indicates that BCMA CAR31-T cells can be effectively activated, further confirming that BCMA CAR31-T cells have more powerful anti-tumor activity. BCMA CAR31-T has a strong in vitro proliferation ability. In order to evaluate the proliferation ability of BCMA CAR31-T cells in vitro, we used a CFSE-based detection method to measure proliferation, and the Pan-T group BCMA CAR16-T group was used as a control. The results are shown in FIG. 18: after 72 hours of cell culture, the CFSE green fluorescence signal of BCMA CAR16-T cells was significantly weakened compared with the BCMA CAR31-T group cells, indicating that the BCMA CAR31-T cells proliferated faster.

### Example 8 Optimization of BCMA CAR-T In Vivo Anti-Tumor Function Verification

It was found that BCMA CAR31-T cells are highly efficient and specific in killing tumor cells, and have strong proliferation and cytokine secretion capabilities in vitro. The xenograft tumor model was further used to verify the anti-tumor ability of BCMA CAR31-T cells in vivo. The experimental scheme is shown in FIG. 19.

### I. Successful Establishment of Mouse Xenograft Tumor Model

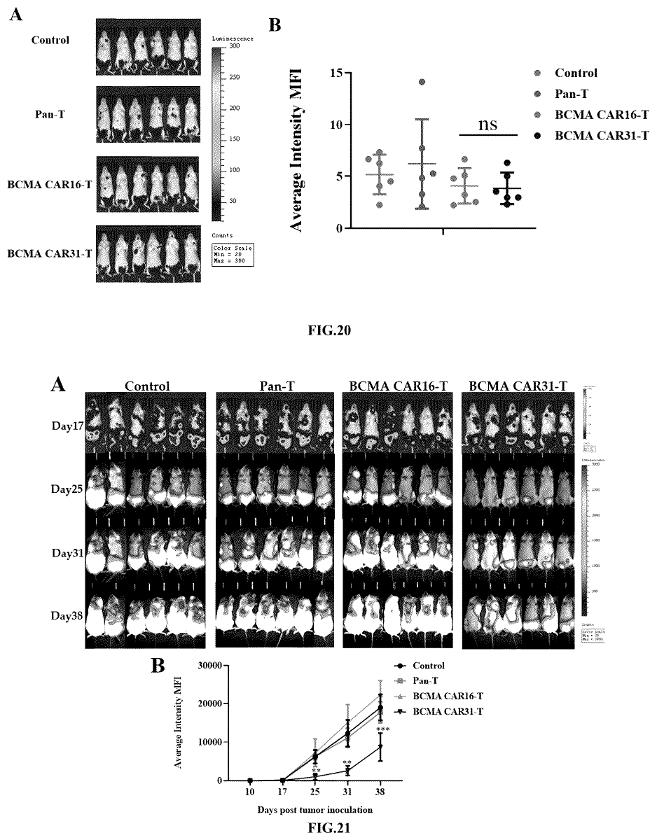

NPG mice were injected with 2×10⁶ RPMI-gfp-luc cells via the tail vein. Ten days later, mice were imaged in vivo. The results are shown in FIG. 20: tumor signals were visible in all mice, and the tumors were uniform, indicating that a mouse xenograft tumor model was successfully established. The mice were randomly divided into four groups. There was no statistical difference in tumor signals between the BCMA CAR31-T group and the BCMA CAR16-T group.

### II. BCMA CAR-T Cells can Effectively Fight Tumors In Vivo

Starting from the sixth day after the first BCMA CAR-T injection, in vivo imaging of mice was performed once a week to detect changes in mouse tumor signal intensity. The results are shown in FIG. 21: after the first injection of BCMA CAR31-T, there was no statistical difference in the mouse tumor signal between BCMA CAR16-T and BCMA CAR16-T. After the second injection, mouse imaging showed that the tumor signal in the BCMA CAR31-T group was significantly weakened compared with BCMA CAR16-T, and the difference was statistically significant. This result shows that BCMA CAR31-T has effective anti-tumor ability in vivo.

### III. BCMA CAR-T Secretes Increased IFN-γ

48 h after the two BCMA CAR-T cell injections, peripheral blood of mice was collected, serum was separated, and ELISA was used to detect the secretion level of human IFN-γ in serum. The results are as follows: after the first BCMA CAR-T injection, the IFN-γ secretion level of BCMA CAR31-T was low at 180.29±31.63 pg/mL, which was no different from BCMA CAR16-T (157.74±45.51 pg/mL), and the results were consistent with the above-mentioned mouse in vivo imaging results, with no obvious anti-tumor effect. After the second BCMA CAR-T injection, compared with BCMA CAR16-T (1254.03±1523.75 pg/mL), the IFN-γ secretion level of BCMA CAR31-T was significantly increased (3778.37±934.68 pg/mL), and the results were consistent with the above-mentioned mouse in vivo imaging, with significant anti-tumor effect. The results of this study showed that BCMA CAR31-T was effectively activated after the second CAR-T injection.

### IV. BCMA CAR31-T has a Lasting Anti-Tumor Effect In Vivo

Blood was collected on the 24th, 31st and 37th days after the mice were inoculated with tumor cells, and the injected CD3+ T cells in the peripheral blood of the mice were detected. The results are as follows: on the 24th day after tumor cell inoculation, the content of CD3+ T cells in each 100 μL of peripheral blood of mice was (−1474.00±3670.66) in the model group, (−288.33±8110.37) in the Pan-T group, (11800.00±7019.97) in the BCMA CAR16-T group, and (29850.00±15939.23) in the BCMA CAR31-T group, the BCMA CAR31-T group in the peripheral blood of mice was significantly higher than that in the BCMA CAR16-T group, the difference was statistically significant; on the 31st day after tumor cell inoculation, the content of CD3+ T cells in each 100 μL mouse peripheral blood was, model group (−4996.00±2067.62), Pan-T group (−983.33±1922.31), BCMA CAR16-T group (8000.00±7186.93), BCMA CAR31-T group (23800.00±11440.80), the content of BCMA CAR31-T group in the peripheral blood of mice was significantly higher than that of BCMA CAR16-T group, the difference was statistically significant; on the 37th day after tumor cell inoculation, the content of CD3+ T cells in the peripheral blood of mice per 100 μL was (1783.33±8431.47) in the model group, (−1750.00±3573.65) in the Pan-T group, (11400.00±9017.98) in the BCMA CAR16-T group, and (54866.67±18619.20) in the BCMA CAR31-T group. The BCMA CAR16-T group content in the peripheral blood of mice in the was significantly higher than that in the BCMA CAR16-T group, and the difference was statistically significant. The results showed that compared with the BCMA CAR16-T group, the BCMA CAR31-T group survived longer in vivo and showed a more persistent anti-tumor ability.

Those skilled in the art will also recognize, or be able to ascertain using no more than routine experimentation, many equivalents to the specific embodiments of the invention described herein. Such equivalents are also intended to be encompassed by the appended claims.

1. A BCMA-targeting chimeric antigen receptor, wherein the receptor comprises a humanized BCMA-targeting ScFv structure, wherein the heavy chain variable region ScFv-VH and the light chain variable region ScFv-VL of the ScFv structure are connected by one or more G4S sequences; the amino acid sequence of the heavy chain variable region ScFv-VH has at least 90% homology with the following SEQ ID NO.8, preferably at least 95% homology, and more preferably at least 98% homology, SEQ ID NO.8:
SQVTLRESGPGLVRPSQTLSLTCTVSGGSIDSGGHYWSWIRQHPG
KGLEWIGSIYHSGNTYYNPSLKSRVTMSVDTSKNQFSLKLTSVTA
ADTAIYYCARDIPHYFEPAYWGQGTLVTVSS;
the amino acid sequence of the light chain variable region ScFv-VL has at least 90% homology with the following SEQ ID NO.9, preferably at least 95% homology, and more preferably at least 98% homology: SEQ ID NO.9:
QSALTQPASASGSPGQSVTISCTGTSSDVGGYNYVSWYQQHPGKA
PKLMIYEVSNRPSGVSNRFSGSKSGNTASLTISGLQAEDEADYYC
AIWHSSAWVFGGGTKLTVLG.

2. The BCMA-targeting chimeric antigen receptor according to claim 1, wherein the humanized BCMA-targeting ScFv structure of the receptor is ScFv-VH-(G4S) n-ScFv-VL, wherein n is an integer greater than or equal to 1, preferably 3 or 4.

3. The BCMA-targeting chimeric antigen receptor according to claim 1, wherein the humanized BCMA-targeting ScFv structure of the receptor is ScFv-VH-(G4S) 3-ScFv-VL, the amino acid sequence of the heavy chain variable region ScFv-VH is SEQ ID NO.8; the amino acid sequence of the light chain variable region ScFv-VL is SEQ ID NO.9.

4. The BCMA-targeting chimeric antigen receptor according to claim 1, wherein the humanized BCMA-targeting ScFv structure of the receptor is ScFv-VH-(G4S) n-ScFv-VL-(G4S) n-ScFv-VL-(G4S) n-ScFv-VH, wherein n is an integer greater than or equal to 1, preferably 3 or 4.

5. The BCMA-targeting chimeric antigen receptor according to claim 1, wherein the humanized BCMA-targeting ScFv structure of the receptor is ScFv-VH-(G4S) 3-ScFv-VL-(G4S) 4-ScFv-VL-(G4S) 3-ScFv-VH, the amino acid sequence of the heavy chain variable region ScFv-VH is SEQ ID NO.8; the amino acid sequence of the light chain variable region ScFv-VL is SEQ ID NO.9.

6. The BCMA-targeting chimeric antigen receptor according to claim 1, wherein the receptor comprises an upstream signal peptide and a myc tag for detection; a humanized BCMA-targeting ScFv structure comprising a heavy chain variable region and a light chain variable region; a CD8 hinge-transmembrane domain; a CD28 or 4-1BB synergistic activation domain and a CD3ζ intracellular signaling domain, which are sequential tandem.

7. A BCMA-targeting chimeric antigen receptor T cell, wherein the T cell expresses the chimeric antigen receptor of claim 1.

8. A drug for treating tumors, wherein the drug comprises the chimeric antigen receptor T cell according to claim 7.

9. A method of treating tumor in a subject, comprising administering to the subject a T cell that expresses the chimeric antigen receptor according to claim 1.

10. The method according to of claim 9, wherein the tumor is a surface BCMA-positive tumor.

11. The method according to of claim 9, wherein the tumor is multiple myeloma.

12. A method of preparing T cells for treating a surface BCMA-positive tumor, comprising infecting human T cells with viral particles made from a viral vector comprising a nucleic acid encoding the chimeric antigen receptor of claim 1, thereby obtaining chimeric antigen receptor T cells for the treatment of surface BCMA-positive tumors.
